(19) United States
(12) Patent Application Publication
(10) Pub. No.: US 2022/0152012 A1
(43) Pub. Date: May 19, 2022

(54) METHODS FOR REGRESSING OR REVERSING FIBROSIS AND/OR LIVER CIRRHOSIS IN A SUBJECT IN NEED THEREOF USING HIGH-DOSE NIACIN, OR A NIACIN ANALOG THEREOF

(71) Applicants: The Regents of the University of California, Oakland, CA (US); United States Government As Represented By The Department of Veterans Affairs, Washington, DC (US)

(72) Inventors: Moti Lal Kashyap, Rancho Palos Verdes, CA (US); Vaijinath Kamanna, Irvine, CA (US); Shobha Kamanna, Irvine, CA (US)

(21) Appl. No.: 17/528,173

(22) Filed: Nov. 16, 2021

Related U.S. Application Data
(60) Provisional application No. 63/114,494, filed on Nov. 16, 2020.

(51) Int. Cl. A61K 31/455; A61P 1/16; A61K 45/06

(52) U.S. Cl. CPC A61K 31/455; A61K 45/06; A61P 1/16

(57) ABSTRACT

The disclosure provides methods to reverse or regress fibrosis and/or liver cirrhosis in a subject in need thereof, using high-dose (pharmacologic) niacin, or a niacin analog thereof.

### CROSS REFERENCE TO RELATED APPLICATIONS

This application claims priority under 35 U.S.C. § 119 from Provisional Application Ser. No. 63/114,494, filed Nov. 16, 2020, the disclosure of which is incorporated herein by reference.

### TECHNICAL FIELD

The disclosure provides methods to reverse or regress fibrosis and/or liver cirrhosis in a subject in need thereof, using high-dose (pharmacologic) niacin, or a niacin analog thereof.

### BACKGROUND

Hepatic fibrosis, a wound healing response of the liver, is mainly caused by chronic liver injuries with diverse etiologies including NASH, viral hepatitis, alcoholism and autoimmune liver diseases. The hallmark of hepatic fibrosis is the excessive accumulation of extracellular matrix proteins (e.g., collagen type 1) produced by activated hepatic stellate cells. Stellate cells are quiescent in normal liver, but upon activation by liver injury these activated hepatic stellate cells are the primary cell types producing extracellular matrix proteins, leading to hepatic fibrosis. Increased hepatic oxidative stress and generation of reactive oxygen species (ROS, an index of oxidative stress) play a crucial role in stellate cell activation and hepatic fibrosis. Hepatic fibrosis with persistent deposition of collagen results in distortion of hepatic parenchyma and vascular structure, clinically manifesting as liver cirrhosis.

### SUMMARY

Collagen deposition is a major underlying histologic feature in hepatic fibrosis and manifests clinically as liver cirrhosis and its complications. Niacin (not as vitamin but as drug) is an anti-dyslipidemic drug used for treatment of atherosclerotic cardiovascular disease. Niacin's action on human hepatic fibrosis is unknown. The efficacy of niacin on regression of preexisting collagen content and changes in oxidative stress were studied herein in cultured primary human hepatic stellate cells. The cells were selected from fresh livers of recently deceased patients with histologic fibrosis and associated steatosis and inflammation, and in subjects without fibrosis. Collagen content in stellate cells from patients were 4-fold higher than in cells from non-fibrosis subjects. Treatment of stellate cells with pharmacologically relevant concentrations of niacin (i.e., high-dose niacin) produced a significant dose and time dependent decrease in pre-existing collagen by 48-60% at 48 h incubation and 54-65% at 96 h incubation. In stellate cells from non-fibrosis subjects, niacin prevented, and suppressed collagen formation induced by oxidative stressors TGF-β and hydrogen peroxide. Pharmacologic doses of niacin significantly inhibited production of reactive oxygen species induced by oxidative stressors: palmitic acid or hydrogen peroxide, by 52% and 50% respectively. Accordingly, the data presented herein demonstrated that pharmacologic doses of niacin regressed or reversed preexisting fibrosis in primary human stellate cells likely via oxidative stress reduction. As liver fibrosis manifests clinically as cirrhosis, pharmacologic doses of niacin, or a niacin analog thereof, can be used for the treatment of cirrhosis of the liver.

In a particular embodiment, the disclosure provides a method to reverse or regress fibrosis and/or liver cirrhosis in a subject in need thereof, comprising: administering to a subject having fibrosis and/or liver cirrhosis one or more pharmaceutical doses of a pharmaceutical composition comprising niacin or of a niacin analog thereof, wherein the pharmaceutical composition comprise 250 mg to 2000 mg of niacin, or niacin equivalent dosing of a niacin analog thereof, wherein the subject is administered a total daily dose of 250 mg to 6000 mg of niacin, or niacin equivalent dosing of a niacin analog thereof, and wherein administration of the one or more pharmaceutical doses of niacin or a niacin analog thereof reverses or regresses fibrosis and/or liver cirrhosis in the subject. In a further embodiment, the fibrosis is associated with elevated or overaccumulation of collagen in cells or tissue. In another or a further embodiment herein, administration of one or more pharmaceutical doses of niacin or of a niacin analog to the subject reduces collagen levels in fibrotic tissue. In another or a further embodiment herein, administration of one or more pharmaceutical doses of niacin or of a niacin analog stabilizes or normalizes the expression levels of matrix metalloproteinases (MMPs) and/or tissue inhibitors of metalloproteinases (TIMPs). In another or a further embodiment herein, the fibrosis affects one or more tissues or organs. In another or a further embodiment herein, the one or more tissues or organs are selected from liver, bone marrow, lung, kidney, gastrointestinal tract, skin, eye, endomyocardium, musculoskeletal system, and myocardium. In another or a further embodiment herein, the one or more tissues or organs is the liver. In another or a further embodiment herein, the subject has a disease, disorder, or condition selected from the group consisting of a cystic fibrosis, idiopathic pulmonary fibrosis, post COVID-19 fibrosis, radiation-induced lung injury, liver fibrosis, liver cirrhosis, glial scars, arterial stiffness, arthrofibrosis, Crohn's disease, Dupuytren's contracture, keloids, mediastinal fibrosis, myelofibrosis, Peyronie's disease, nephrogenic system fibrosis, progressive massive fibrosis, retroperitoneal fibrosis, scleroderma/systemic sclerosis, adhesive capsulitis, interstitial fibrosis, replacement fibrosis, inflammatory bowel disease, renal fibrosis in patients with tubulointerstitial fibrosis, glomerulosclerosis, lung fibrosis, and chronic kidney disease. In another or a further embodiment herein, the subject has grade 1, grade 2, grade 3, or grade 4 liver fibrosis. In another or a further embodiment herein, the subject has grade 1, grade 2, or grade 3 liver fibrosis. In another or a further embodiment herein, the subject has liver cirrhosis. In another or a further embodiment herein, the subject has liver fibrosis and nonalcoholic steatohepatitis, or liver fibrosis and alcoholic steatohepatitis. In another or a further embodiment herein, the subject has liver fibrosis resulting from a biliary obstruction, iron overload, autoimmune hepatitis, Wilson's disease, a viral hepatitis B infection, or a viral hepatitis C infection. In another or a further embodiment herein, the niacin analog is selected from nicotinamide, 6-hydroxy nicotinamide, N-methyl-nicotinamide, acifran, acipimox, niceritrol, ARI-3037MO, and nicotinamide riboside chloride. In another or a further embodiment herein, the pharmaceutical composition is formulated for oral, transdermal or parenteral delivery. In another or a further embodiment herein, the pharmaceutical composition is formulated as an extended-release or time-release formulation for oral delivery. In another or a further embodiment herein, the pharmaceutical composition is formulated as a film-coated extended-release tablet. In another or a further embodiment herein, the film-coated extended-release tablet comprises hypromellose, povidone, stearic acid, polyethylene glycol, and/or coloring reagents. In another or a further embodiment herein, the pharmaceutical composition is formulated as a tablet and comprises croscarmellose sodium, hydrogenated vegetable oil, magnesium stearate and/or microcrystalline cellulose. In another or a further embodiment herein, the one or more pharmaceutical doses are administered sequentially or concurrently with one or more therapeutics selected from anti-fibrotic therapeutics, prostaglandin D2 binding drugs, antivirals, gallstone solubilizing agents, anti-thrombotic treatments, nonalcoholic fatty liver disease (NAFLD) treatments, nonalcoholic steatohepatitis (NASH) treatments, sepsis treatments, anti-mycobacterial agents, chelation therapy agents, anti-bacterial agents, anti-fungal agents, steroidal drugs, anticoagulants, non-steroidal anti-inflammatory agents, antiplatelet agents, norepinephrine reuptake inhibitors (NRIs), dopamine reuptake inhibitors (DRIs), Serotonin and norepinephrine reuptake inhibitors (SNRIs), sedatives, Norepinephrine and Dopamine Reuptake Inhibitors (NDRIs), serotonin-norepinephrine-dopamine reuptake inhibitors (SNDRIs), monoamine oxidase inhibitors, hypothalamic phospholipids, Endothelin converting enzymes (ECE) inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIa Inhibitors and Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, microsomal triglyceride transfer protein (MTP) Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, anti-inflammatories, anti-proliferatives, chemotherapeutic agents, immunosuppressants, anticancer agents and cytotoxic agents, anti-metabolites, antibiotics, farnesyl-protein transferase inhibitors, hormonal agents, microtubule-disruptor agents, microtubule-stabilizing agents, plant-derived products, epipodophyllotoxins, taxanes, topoisomerase inhibitors, prenyl-protein transferase inhibitors, cyclosporins, cytotoxic drugs, tumor necrosis factor (TNF)-alpha inhibitors, anti-TNF antibodies and soluble TNF receptors, cyclooxygenase-2 (COX-2) inhibitors, galectin inhibitors, transforming growth factor (TGF)-beta inhibitors, anti-TGF-beta antibodies, anti-oxidants, oxidative stress inhibitors, TIMP inhibitors, matrix metalloproteinase-1 (MMP) activators, kynurenic acid, FS2, cenicriviroc, aramchol, aramchol meglumine, and belapectin. In another or a further embodiment herein, the one or more pharmaceutical doses are administered sequentially or concurrently with one or more anti-fibrotic therapeutics. In another or a further embodiment herein, the one or more anti-fibrotic therapeutics are selected from nintedanib, pirfenidone, rilonacept, tocilizumab, rituximab, abatacept, lanifibranor, NCT02503644, NCT03597933, FCX-103, and SAR100842. In another or a further embodiment herein, the one or more pharmaceutical doses is administered sequentially or concurrently with a prostaglandin D2 binding drug. In another or a further embodiment herein, the prostaglandin D2 binding drug is laropiprant. In another or a further embodiment herein, the one or more pharmaceutical doses are administered sequentially or concurrently with one or more therapeutics selected from NASH treatments, NAFLD treatments, antiviral drugs, and gallstone solubilizing agents. In another or a further embodiment herein, the NASH treatments and NAFLD treatments are selected from orlistat, elafibranor, pioglitazone, saroglitazar, solithromycin, exenatide, liraglutide, sitagliptin, vildapliptin, aramchol, obeticholic acid, cenicriviroc, pentoxifylline, emricasan, simtuzumab, galectin-3, atorvastatin, pravastatin, cerivastatin, lovastatin, mevastatin, pitavastatin, rosuvastatin, simvastatin, fluvastatin, NGM-282, GS-4997, IMM-124e, cysteamine, cystamine, and vitamin E. In another or a further embodiment herein, the one or more pharmaceutical doses are administered sequentially or concurrently with one or more therapeutics that target lipid metabolism and insulin resistance; one or more therapeutics that target lipotoxicity, oxidative stress, and inflammation; one or more therapeutics that target fibrosis and cirrhosis; or a combination thereof. In another or a further embodiment herein, the one or more pharmaceutical doses are administered sequentially or concurrently with one or more therapeutics selected from acetyl-CoA carboxylase (ACC) Inhibitors, fatty acid synthase inhibitors, icosbutate, eicosapentaenoic acid analogs/derivatives, omega/n-3 fatty acids, thiazolidinediones (TZDs), 3-hydroxy-3-methylglutaryl-coenzyme A reductase (HMG-CoA reductase) inhibitors, dibrates, peroxisome proliferated-activated receptors (PPAR)-alpha/beta/gamma/delta agonists/modulators, fibroblast growth factor (FGF)-19/21 analogs/modulators, glucagon-like peptide receptors (GLP-1) analogs/mimetics/modulators, ketohexokinase inhibitors, mitochondrial pyruvate carrier inhibitor/modulators, sodium-glucose cotransporters (SGLTs) inhibitors/modulators, adenosine monophosphate-activated protein kinase (AMPK) activators/modulators, stearoyl-CoA dehydrogenase (SCD) inhibitors, diacylglycerol-acyl transferase-2 (DGAT-2) inhibitors, thyroid hormone receptor-beta (THR-beta) agonists, glucocorticoid modulators, dipeptidyl peptidase-4 inhibitors (DPP-4), sodium glucose co-transporter 2 (SGLT2) inhibitors, anti-diabetes 2 agents, PPAR-alpha/beta/gamma/delta agonists/modulators, farnesoid X receptor (FXR) agonists/modulators, vitamin E, anti-oxidants, FGF-19/21 modulators, chemokine 2/5 receptor (CCR2/5) antagonists/Inhibitors, nicotinamide adenine dinucleotide phosphate oxidase (NADPH oxidase) inhibitors, and angiotensin receptor antagonists, caspases inhibitors/modulators, apoptosis inhibitors/modulators, leukotriene/phosphodiesterase/lipoxygenase antagonists/inhibitors/modulators, galectin-3 antagonists/modulators, apoptosis signal-regulating kinases (ASK) inhibitors, lysophosphatidic acid receptor 1 (LPA1) antagonists, and heat shock proteins (HSP47 and other members) inhibitors. In another or a further embodiment herein, the one or more pharmaceutical doses are administered sequentially or concurrently with one or more therapeutics selected from laropiprant, cenicriviroc, resmetirom, ocaliva, elafibranor, aramchol, IMM124E, semaglutide, lanifibranor, seladelpar, belapectin, PXL_065, MADC_0602, aldafermin, VK2809, EDP_305, PF_05221304, tipelukast, tropifexor, DF102, LMB763, nitazoxanide, tesamorelin, TERN_101, lazarotide, BMS986036, Saroglitazar, AKR001, CRV431, GRI_0621, EYP001, BMS_986171, isosabutate, PF_06835919, PF_06865571, nalmefene, LIK066, BIO89_100, Namodenoson, MT_3995, pemafibrate, PXL770, gemcabene, foralumab, SGM_1019, KBP_042, hepastem, CER_209, DUR928, sotagliflozin, elobixibat, SAR425899, NGM313, namacizumab, TERN_201, LPCN_1144, ND_L02 S0201, RTU_1096, IONIS_DGAT2R, bezafibrate, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, and GKT_137831.

### DESCRIPTION OF DRAWINGS

FIG. 1A-B demonstrates that niacin decreased fibrosis in hepatic stellate cells from human donor subjects with fibrotic NASH (Donors 3-7): Human hepatic stellate cells from donor subjects with varying degree of fibrosis and NASH (NAFLD activity scores 2-5, and fibrosis scores 1-3) were incubated with pharmacologically relevant concentrations of niacin (0.25 mM and 0.5 mM) for 48 or 96 hours. Cells were stained with Sirius Red for collagen content, cellular photographic images, and quantitation. (A) Representative photographic images of stellate cells (left to right): “Non-NASH” from donor 1, “NASH” from donor 6 without niacin, with 0.5 mM niacin at 48, and 96 h incubation. (B) Composite Mean±SE collagen content data from all 5 NASH-Fibrosis patient (donors 3-7) showing the ability of niacin to regress pre-existing fibrosis in stellate cells from patients with NASH and fibrosis. Left bar marked “Normal” refers to mean collagen content in stellate cells from non-NASH subjects (donors 1, 2). Right 3 bars refer to stellate cells from NASH patients (donors 3-7) with fibrosis treated with niacin at 0, 0.25, and 0.5 mM. *, p<0.001 vs 0 mM Niacin; a, p<0.0001 vs Normal (Non-NASH subject); b, p<0.03 vs 0.25 mM Niacin.

FIG. 2 shows that niacin prevented TGF-β or H₂O₂-induced collagen production in human hepatic stellate cells from human subjects without liver fibrosis. Cells were incubated with TGF-β (20 ng/mL) or H₂O₂ (25 μM) in the absence or presence of niacin (0.5 mM) for 24 h. Cells were stained with Sirius Red for assessment of collagen content. Top Panel: Representative cellular photographic images of collagen deposition. Bottom Panel: Quantitation of collagen content in stellate cells. VEH, PBS vehicle, NIA, treatment with niacin (0.5 mM) alone without H₂O₂ or TGF-β. *, p<0.05 vs VEH; +, p<0.05 vs respective H₂O₂ or TGF-β.

FIG. 3 demonstrates that niacin reversed hepatic stellate cell fibrosis induced by TGF-β or H₂O₂ in human hepatic stellate cells from normal human donor subjects without liver fibrosis. Human hepatic stellate cells from control subjects without liver fibrosis were first stimulated with TGF-β (20 ng/mL) or H₂O₂ (25 μM) for 24 h to induce collagen deposition. These cells were then continued to incubate additional 24 h in the absence or presence of niacin (0.5 mM). The cellular content of Collagen type I was assessed. Control, no prior treatment with H₂O₂ or TGF-β; Niacin 0.5 mM, treatment with only niacin without prior treatment with H₂O₂ or TGF-β *, p<0.05 vs Control; +, p<0.05 vs respective H₂O₂ or TGF-β.

FIG. 4 demonstrates that niacin prevented human hepatic stellate cell oxidative stress induced by palmitic acid or H₂O₂. Cells from subjects without liver fibrosis were first stimulated with either palmitic acid (0.5 mM) or H₂O₂ (25 μM) for 24 h to induce oxidative stress. These cells were incubated an additional 24 h in the absence or presence of niacin (0.5 mM). The ROS levels were then measured. Top Panel: Representative cellular photographic images after staining with DCFDA. Bottom Panel: Quantitative levels of ROS presented as percent control, VEH, PBS vehicle alone, NIA, treatment with niacin (0.5 mM) alone without H₂O₂ or palmitic acid. PA or PAL, palmitic acid. *, p<0.05 vs VEH; +, p<0.05 vs Palmitic acid or H₂O₂, respectively.

FIG. 5 provides that niacin had no effect on human hepatic stellate cell viability. Cells were incubated with niacin (0-0.5 mM) for 0 h, 48 h, or 96 h. Cellular viability was assessed. NIA, niacin.

FIG. 6 diagrams a possible mechanism of action of niacin on liver fibrosis resolution. Based on the results presented herein, it postulated that reduction of oxidative stress is a major route for niacin's effect on fibrosis and hence improvement in cirrhosis. By inhibiting NADPH oxidase and glutathione peroxidase, hepatic oxidative stress is reduced resulting in fibrosis mitigation by 2 major pathways: (1) reduced lipotoxicity deactivates stellate cells thereby leading to reductions in TGF-β and its amplifying CTGF-mediated signaling events, and a reduction in collagen production; and (2) a reduction in oxidative stress leading to a decrease in TIMP activity and an increase in MMP activity. Thus, it is proposed that niacin also reduces the TIMP/MMP ratio resulting in accelerated degradation of collagen. The combined effect these 2 pathways is fibrosis resolution. *Demonstrated in animal NASH fibrosis model and in human stellate cell model. α-SMA: α-smooth muscle cell actin, TGF-β: transforming growth factor-4, CTGF: connective tissue growth factor, TIMP: tissue inhibitor of metalloproteinase, MMP: matrix metalloproteinase

### DETAILED DESCRIPTION

As used herein and in the appended claims, the singular forms “a”, “an”, and “the” include plural referents unless the context clearly dictates otherwise. Thus, for example, reference to “an extracellular matrix protein” includes a plurality of such extracellular matrix proteins and reference to “the liver disease” includes reference to one or more liver diseases thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in their entirety for the purposes of describing and disclosing methodologies that might be used in connection with the description herein. Moreover, with respect to any term that is presented in the publications that is similar to, or identical with, a term that has been expressly defined in this disclosure, the definition of the term as expressly provided in this disclosure will control in all respects.

Unless defined otherwise, all technical and scientific terms used herein have the same meaning as commonly understood by one of ordinary skill in the art to which this disclosure belongs. Although many methods and reagents similar to or equivalent to those described herein can be used in the practice of the disclosed methods and compositions, the exemplary methods and materials are now described.

The term “about”, as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “disorder” as used herein is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

The term “high-dose niacin” or “a high dose of niacin” refers herein to a dose of niacin or a niacin analog thereof that is from 250 mg to 2000 mg of niacin, or niacin equivalent dosing of a niacin analog thereof “High-dose niacin” can include a pharmaceutical dose of niacin, or a dietary dose of niacin.

The term “pharmaceutical dose of niacin” as used herein refers to a dose of niacin or a niacin analog thereof that is effective to reverse or regress fibrosis and/or liver cirrhosis in a subject. A “pharmaceutical dose of niacin” greatly exceeds the daily recommended dietary allowance (20 mg) of niacin. For purposes of this disclosure, a “pharmaceutical dose of niacin” refers to a dose of niacin or niacin analog thereof that is under the care and monitoring of a healthcare provider. Accordingly, a “pharmaceutical dose of niacin” constitutes a pharmaceutical use, not a dietary supplement use.

The term “niacin analog” refers to a compound that has a structure that may differ from niacin but when administered to a subject elucidates a similar response as niacin. A “niacin analog” includes prodrugs of niacin, niacin metabolites, mimetics of niacin, and derivatives of niacin. Specific examples of “niacin analogs” include, but are not limited to, acifran, acipimox, niceritrol, isonicotinic acid, isonicotinic hydrazide, 3-pyridine acetic acid, 5-methylnicotinic acid, pyridazine-4-carboxylic acid, pyrazine-2-carboxylic acid, ARI-3037MO, 3-pyridylcarbinol, 3-acetylpryidine, and nicotinamide riboside chloride. Further examples of “niacin derivatives”, “niacin analogues”, and “niacin mimetics” can be found in the following patent application, which are incorporated in full herein: US20160151343A1, U.S. Pat. No. 9,511,060B2, U.S. Pat. No. 9,212,142B2, U.S. Pat. No. 8,937,063B2, WO2005102331A1, RU2588133C2, AU2015203711A1, WO2012175049A1, CN103096895B CA2659747C, AU2005272043B2, JP2008518957A, and JP2008520715A. In a particular embodiment, the term “niacin analog” does not include inositol hexanicotinate (IHN).

The term “niacin metabolite” or “metabolite of niacin” as used herein refers to a metabolite generated from metabolism of niacin by an organism, particularly a mammalian organism. Specific examples of “niacin metabolites” include, but are not limited to, nicotinuric acid, nicotinamide, 6-hydroxy nicotinamide, N-methyl-nicotinamide, nicotinamide-N-oxide, N-methyl-2-pyridone-5-carboxamide, and N-methyl-4-pyridone-5-carboxamide.

The terms “treat”, “treating”, and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention.

The terms “prevent”, “preventing”, and “prevention” refer to a method of delaying or precluding the onset of a disorder; and/or its attendant symptoms, barring a subject from acquiring a disorder or reducing a subject's risk of acquiring a disorder.

The terms “reversing fibrosis”, “regressing fibrosis”, or “resolving fibrosis” are used interchangeably herein, and refer to a process in which the accumulation of extracellular matrix proteins (e.g., collagen) in cells is reduced or suppressed, and/or where the levels of extracellular matrix proteins in fibrotic tissues or organs are decreased or degraded.

The terms “reversing liver fibrosis”, “regressing liver fibrosis”, or “resolving liver fibrosis” are used interchangeably herein, and refer to a process in which the accumulation of extracellular matrix proteins (e.g., collagen) in stellate cells is reduced or suppressed, and/or where the levels of extracellular matrix proteins in fibrotic liver tissue or the liver itself is decreased or degraded.

The term “therapeutically effective amount” refers to the amount of a compound (e.g., high-dose niacin, or a niacin analog thereof) that, when administered, is sufficient to prevent development of, or alleviate to some extent, one or more of the symptoms of the disorder being treated (e.g., fibrosis). The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents (e.g., high-dose niacin, or a niacin analog thereof, and anti-thrombotic) to treat a therapeutic disorder described in the present disclosure. Such administration encompasses co-administration of these therapeutic agents in a substantially simultaneous manner, such as in a single capsule or tablet having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

The term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The terms “active ingredient”, “active compound”, and “active substance” refer to a compound, which is administered, alone or in combination with one or more pharmaceutically acceptable excipients or carriers, to a subject for treating, preventing, or ameliorating one or more symptoms of a disorder. In a particular embodiment, high-dose niacin, or a niacin analog thereof, is an active ingredient in a composition, such as a pharmaceutical composition.

The terms “drug”, or “therapeutic agent”, refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “non-release controlling excipient” refers to an excipient whose primary function do not include modifying the duration or place of release of the active substance from a dosage form as compared with a conventional immediate release dosage form.

Hepatic fibrosis is a scarring process associated with an increased and altered deposition of extracellular matrix components in the liver. It is caused by a variety of stimuli and if fibrosis continues unopposed, it would progress to cirrhosis which poses a significant health problem worldwide. Liver fibrosis is initiated by a cascade of events resulting in hepatocyte damage, recruitment of inflammatory cells to the injured liver, and activation of collagen-producing cells. Hepatic stellate cells (HSC) are a major source of collagen type 1. The fibrogenic response is a complex process in which accumulation of extracellular matrix proteins, tissue contraction, and alteration in blood flow are prominent. Progressive scarring in response to a persisting liver insult eventually results in cirrhosis which is one of the leading causes of death worldwide and a major global health burden. At the cellular and molecular level, this progressive process is characterized by cellular activation of hepatic stellate cells and aberrant activity of transforming growth factor-0 with its downstream cellular mediators. Stellate cells are quiescent in normal liver, but upon activation by liver injury these activated hepatic stellate cells characterized by α-smooth cell actin are the primary cell types producing extracellular matrix proteins leading to hepatic fibrosis. Increased hepatic oxidative stress and generation of reactive oxygen species (ROS, an index of oxidative stress) play a crucial role in stellate cell activation and hepatic fibrosis. Hepatic fibrosis with persistent deposition of collagen results in distortion of hepatic parenchyma and vascular structure, clinically manifesting as liver cirrhosis and with fatal complications.

Liver fibrosis results from perpetuation of the normal wound healing response, resulting in an abnormal continuation of fibrogenesis. It is characterized by an excessive deposition of extracellular matrix (ECM) proteins which includes three large families of proteins—glycoproteins, collagens, and proteoglycans. Fibrosis occurs as a result of repeated cycles of hepatocytes injury and repair. The cascade of events that establish hepatic fibrosis is complex, and is influenced by how different cell types in the liver interact in response to injury, and activation of HSC is the central event. Liver fibrosis is a dynamic process; it is usually secondary to hepatic injury and inflammation, and progresses at different rates depending on the etiology of liver disease and is also influenced by environmental and genetic factors. If fibrosis continues unopposed, it would disrupt the normal architecture of the liver which alters the normal function of the organ, ultimately leading to pathophysiological damage of the liver. Cirrhosis represents the final stages of fibrosis. It is characterized by fibrous septa which divide the parenchyma into regenerative nodules which leads to vascular modifications and portal hypertension with its complications of variceal bleeding, hepatic encephalopathy, ascites, and hepatorenal syndrome. In addition, this condition is largely associated with hepatocellular carcinoma with a further increase in the relative mortality rate.

Liver cirrhosis is a silent disease and clinical manifestations of portal hypertension from cirrhosis occur after decades of life with serious and costly complications including ascites, esophageal varices and hemorrhage, hepatic encephalopathy, and liver failure. Prolonged cirrhosis has been recognized as a “premalignant state” with increased risk of hepatocellular carcinoma. Thus, prevention in high-risk individuals is as important as treatment. Current management emphasizes lifestyle change (diet and exercise) as the initial treatment. However, this is not always successful and pharmacologic approaches are needed.

The mechanisms able to elicit and sustain liver fibrogenesis may be classified in three main groups: (a) chronic activation of the wound healing reaction, (b) oxidative stress, and (c) derangement of epithelial-mesenchymal interactions and epithelial-mesenchymal transition in cholangiopathies.

Similar to what was observed in other fibrogenic disorders affecting different organs and systems, the chronic activation of the wound-healing reaction is the most common and relevant mechanism in hepatic fibrogenesis. Overall, hepatic fibrogenesis due to the chronic activation of the wound healing reaction is characterized by the following key features: (i) the persistence of hepatocellular/cholangiocellular damage with variable degree of necrosis and apoptosis; (ii) a complex inflammatory infiltrate including mononuclear cells and cells of the immune system; (iii) the activation of different types of ECM-producing cells (HSCs, portal myofibroblasts (MFs), etc.) with marked proliferative, synthetic, and contractile features; and (iv) marked changes in the quality and quantity of the hepatic ECM associated with very limited or absent possibilities of remodeling in the presence of a persistent attempt of hepatic regeneration.

Involvement of oxidative stress has been documented in all human major clinical conditions of chronic liver disease (CLD) as well as in most experimental models of liver fibrogenesis, but it is likely to represent the predominant profibrogenic mechanism mainly in NAFLD/NASH and alcoholic steatohepatitis (ASH). Oxidative stress in CLD, resulting from increased generation of reactive oxygen species (ROS) and other reactive intermediates as well as by decreased efficiency of antioxidant defenses, does not represent simply as a potentially toxic consequence of chronic liver injury but actively contributes to excessive tissue remodeling and fibrogenesis. ROS and other reactive mediators such as 4-hydroxynonenal (HNE) can be generated outside MFs, being released either by activated inflammatory cells or deriving from hepatocytes, directly or indirectly, damaged by the specific etiological agent or conditions. Indeed, oxidative stress, presumably by favoring mitochondrial permeability transition, is able to promote hepatocyte death (necrotic and/or apoptotic). In some of clinically relevant conditions, generation of ROS within hepatocytes may represent a consequence of an altered metabolic state (like in NAFLD and NASH) or of ethanol metabolism (as in ASH), with ROS being generated mainly by mitochondrial electron transport chain or through the involvement of selected cytochrome P₄₅₀ isoforms like cytochrome P2E1 (CYP2E1). Oxidative-stress-related mediators released by damaged or activated neighboring cells can directly affect the behavior of human HSC/MFs: ROS or the reactive aldehyde HNE have been reported to upregulate expression of critical genes related to fibrogenesis including procollagen type I, monocyte chemoattractant protein 1 (MCP-1), and TIMP-1, possibly through activation of a number of critical signal transduction pathways and transcription factors, including activation of c-jun N-terminal kinases (JNKs), transcription factor AP-1 (AP-1) and for ROS, nuclear factor-kB (NF-kB). In addition to ‘profibrogenic’ extracellular release by neighboring cells, ROS generation within human and rat HSC/MFs has been reported to occur in response to several known profibrogenic mediators, including angiotensin II, platelets derived growth factor (PDGF), and the adipokine leptin.

Although several therapeutic targets and drugs have been under investigation, there currently are no FDA approved pharmacological agent(s) for treating liver fibrosis to reduce portal hypertension and cirrhosis complications. It was found herein, that high-dose niacin can be used for the treatment of liver fibrosis and complications thereof. In the studies presented herein high-dose niacin significantly decreased the production of reactive oxygen species (ROS, an index of oxidative stress) via inhibition of NADPH oxidase activity in human hepatocytes and inflammation in various cell types including human aortic endothelium and human blood lymphocytes. It is postulated herein that reduction of oxidative stress and inflammation may be account, at least partially, for fibrosis reduction by niacin.

The studies presented herein demonstrate that niacin not only prevented and reversed collagen deposition in hepatic stellate cells isolated from non-fibrotic livers, but also reversed preexisting collagen deposits (fibrosis) in hepatic stellate cells taken from patients with NASH-fibrosis. The mechanism is by oxidative stress reduction by niacin.

Niacin, also known as nicotinic acid or vitamin B3, is a water-soluble vitamin whose derivatives such as NADH, NAD, NAD⁺, and NADP play essential roles in energy metabolism in the living cell and DNA repair. The designation vitamin B3 also includes the amide form, nicotinamide or niacinamide. Severe lack of niacin causes the deficiency disease pellagra, whereas a mild deficiency slows down the metabolism decreasing cold tolerance. The recommended daily allowance of niacin is 2-12 mg a day for children, 14 mg a day for women, 16 mg a day for men, and 18 mg a day for pregnant or breast-feeding women. It is found in various animal and plant tissues and has pellagra-curative, vasodilating, and antilipemic properties. The liver can synthesize niacin from the essential amino acid tryptophan, but the synthesis is extremely slow and requires vitamin B6; 60 mg of tryptophan are required to make one milligram of niacin. Bacteria in the gut may also perform the conversion but are inefficient.

Both niacin and niacinamide are rapidly absorbed from the stomach and small intestine. Absorption is facilitated by sodium-dependent diffusion, and at higher intakes, via passive diffusion. Unlike some other vitamins, the percent absorbed does not decrease with increasing dose, so that even at amounts of 3-4 grams, absorption is nearly complete. With a one gram dose, peak plasma concentrations of 15 to 30 μg/mL are reached within 30 to 60 minutes. Approximately 88% of an oral pharmacologic dose is eliminated by the kidneys as unchanged niacin or nicotinuric acid, its primary metabolite. The plasma elimination half-life of niacin ranges from 20 to 45 minutes.

After absorption, niacin circulates in the plasma in the unbound form as both the acid and the amide. In the liver, niacinamide is converted to storage nicotinamide adenine dinucleotide (NAD). As needed, liver NAD is hydrolyzed to niacinamide and niacin for transport to tissues. The tissues then reconvert niacinamide and niacin to NAD to serve as an enzyme cofactor. Excess niacin is methylated in the liver to N¹-methylnicotinamide (NMN) and excreted in urine as such or as the oxidized metabolite N¹-methyl-2-pyridone-5-carboxamide (2-pyridone). The main metabolites in humans are N-methylnicotinamide, N-methyl-2-pyridone-5-carboxamide, N-methyl-6-pyridone-3-carboxamide, N-methyl-4-pyridone-3-carboxamide and N-methyl-4-pyridone-5-carboxamide. Decreased urinary content of these metabolites is a measure of niacin deficiency.

Niacin is incorporated into multi-vitamin and sold as a single-ingredient dietary supplement. The latter can be immediate or slow release. One form of dietary supplement sold in the US is inositol hexanicotinate (IHN), also called inositol nicotinate. IHP is made up of inositol that has been esterified with niacin on all six of inositol's alcohol groups. IHN is usually sold as “flush-free” or “no-flush” niacin in units of 250, 500, or 1000 mg/tablets or capsules. In the US, it is sold as an over-the-counter formulation, and often is marketed and labeled as niacin, thus misleading consumers into thinking they are getting an active form of the medication. While this form of niacin does not cause the flushing associated with the immediate-release products, gastrointestinal absorption of inositol hexanicotinate varies widely with an average of 70% of an orally ingested dose absorbed. Once inositol hexanicotinate is present in human serum, hydrolysis of the ester bonds and release of free nicotinic acid is slow, taking more than 48 hours. After oral doses of 0.8 to 4.2 g of inositol hexanicotinate in humans, plasma levels of free nicotinic acid peaks at 6-12 hours. In contrast, after an oral dose of 1000 mg of nicotinic acid, plasma levels of free nicotinic acid peaks at 0.5-1 hour at 30 μg/mL. While IHN is well tolerated, it was found to be no better than placebo for the management of dyslipidemia (see Keenan, Joseph, “Extended-Release Nicotinic Acid Versus Inositol Hexanicotinate for the Treatment of Dyslipidemia.” Journal of Clinical lipidology, 4(3):P216-217 (2010)). IHN does not produce plasma nicotinic acid levels sufficient to lower lipids. The peak plasma levels of nicotinic acid after oral doses of IHN are dramatically lower when compared with those obtained after oral doses of nicotinic acid; for example, a single oral dose of 1,000 mg nicotinic resulted in a peak plasma level of 30 μg/mL nicotinic acid, while 1,000 mg of IHN (weight equivalent to ˜910 mg nicotinic acid) resulted in a peak plasma level of 0.2 μg/mL nicotinic acid (see Harthon et al., “Enzymatic hydrolysis of pentaerythritoltetranicotinate and meso-inositolhexanicotinate in blood and tissues.” Arzneimittelforschung. 29:1859-1862 (1979)).

Quantities of niacin above 500 mg should not be self-administered as a dietary supplement, but may be safely used under the care and monitoring of a healthcare provider. Such an application, it should be noted, constitutes a pharmaceutical use, not a dietary supplement use. Niacin, when used at higher doses, has been used clinically for the treatment of lipid disorders and cardiovascular disease. Pharmacologic doses of niacin have been shown to reduce atherogenic lipids, lipoproteins and several inflammatory markers. As monotherapy, it significantly minimized cardiovascular and stroke events and slowed or reversed occlusive atherosclerosis in combination with LDL-C lowering agents. While niacin has been shown to reverse hepatic steatosis and inflammation, its efficacy on reversing or treating fibrosis in humans is still not known.

Pharmacologic doses of niacin can be immediate release (Niacor, 500 mg tablets) or extended release (Niaspan, 500 and 1000 mg tablets). Niaspan has a film coating that delays release of the niacin, resulting in an absorption over a period of 8-12 hours. Both forms of niacin are considered safe and effective antihyperlipidemic drugs for use under medical supervision and monitoring. Extended-release formulations of niacin substantially reduce the risk of flushing reactions, but carry a greater risk of liver toxicity. ER-NA is approximately twice as hepatotoxic as NA. Combining laropiprant, a prostaglandin D2 binding drug, with niacin leads to a reduction of niacin-induced vasodilation and flushing side effects.

In a preclinical in vitro human model of liver fibrosis, it was shown herein that niacin's efficacy on stellate cell fibrosis reversal and prevention. In the study, primary stellate cells were isolated and cultured in vitro, from fresh livers of patients who had recently died. Patients with histologically diagnosed fibrosis (stage 1 to stage 3) were investigated. Two patients had no fibrosis. As shown (Table 1), these patients were of both genders, wide age range, had comorbidities of varying etiologies including risk factors for NASH, and racial backgrounds. Pharmacological concentrations of niacin (0.25-0.5 mM) used in the in-vitro studies in hepatic stellate cells are clinically relevant and comparable to the niacin concentrations observed in human plasma after oral administration of niacin doses of 1-3 g/daily. Because of the first pass effect via the portal vein, niacin concentration in liver tissue will be much higher than in plasma levels after oral administration of 1-3 g of niacin.

The data in the study demonstrate that niacin (0.25 mM and 0.5 mM) in a dose and time dependent manner markedly (up to 65%) decreased pre-existing fibrosis in stellate cells from patients with varying degrees of fibrotic NASH (NAFLD activity scores 2-5, Fibrosis scores 1-3) in a statistically significant manner. In non-NASH subjects, niacin strikingly prevented, and regressed stellate cell fibrosis induced by major physiological stimulators of liver fibrosis such as inflammatory cytokine TGF-β or oxidative stress mediator H₂O₂.

Of note, in addition to the direct effects of niacin on hepatic fibrosis demonstrated herein, there is also an indirect effect via the mitigating effects of niacin on steatosis and inflammation. By reducing the front-end of this progressive disease, niacin reduced the sequential cascade effect on inflammation and fibrosis. Thus, niacin is a multifactorial therapeutic due to its direct and indirect efficacy on fibrosis reversal and prevention.

Collagen 1 is an important component of liver fibrosis resulting in clinical cirrhosis. The balance between its production and removal determines its content in the liver over time. Oxidative stress and Transforming Growth Factor-beta (TGF-β) play an important role in the pathogenesis, production, and accumulation of hepatic collagen. Briefly, mediators of oxidative stress including NADPH oxidase, Glutathione Peroxidase (GPx), Hydrogen Peroxide (H₂O₂) and saturated fatty acids (e.g., palmitic acid) result in increased lipid peroxidation and activation of hepatocytes and stellate cells as indicated by the marker alpha-Smooth Muscle Actin (α-SMA). The production of TGF-β from oxidative stress has major impact on fibrogenesis. Its known multiple effects include apoptosis in hepatocytes, increased hydrogen peroxide further augmenting oxidative stress in a vicious cycle, and increased production of Connective Tissue Growth Factor (CTGF), a mitogenic protein that amplifies fibrogenesis. These factors result in stimulation of extracellular matrix proteins of which collagen 1 is most abundant. The physiologic removal of hepatic collagen is mediated by macrophages that secrete matrix metalloproteinases (MMP), including MMP 2 and 9. MMPs are regulated by tissue inhibitors of metalloproteinases (TIMPs). The dynamic balance between the MMPs and TIMPs impacts on collagen removal rate. Thus, a decrease in the TIMP/MMP ratio either by increase of MMP or a decrease in TIMP alone or both results in increased collagen removal.

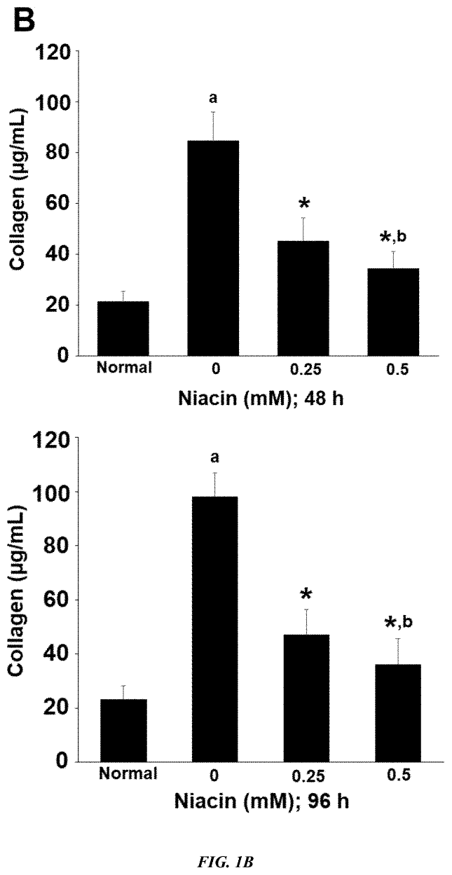

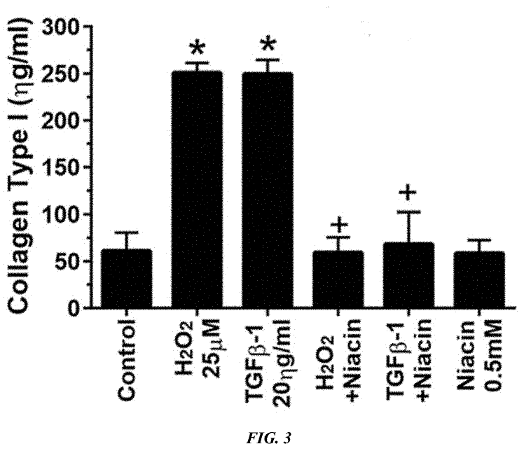

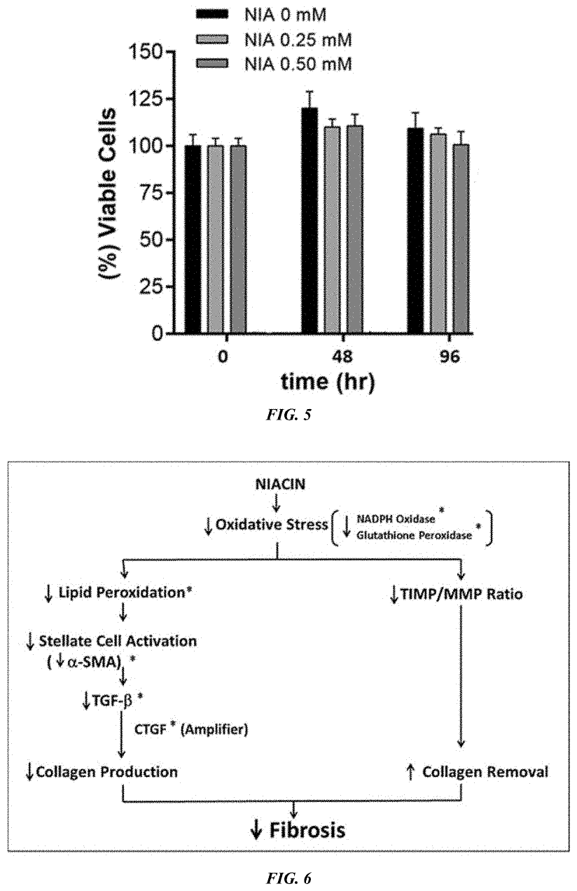

In the studies presented herein, it was shown that high-dose niacin significantly and robustly removed preexisting collagen in cultured stellate cells isolated from a variety of NASH patients with histologically demonstrated fibrosis (grade 1 to grade 3). When human stellate cells, isolated from patients without fibrosis, were challenged with TGF-β or hydrogen peroxide, high-dose niacin suppressed collagen deposition by over 55% (see FIG. 2). High-dose niacin reduced oxidative stress induced by palmitic acid or hydrogen peroxide by over half (see FIG. 4). It is postulated herein that mechanism of action for high-dose niacin is primarily due to a reduction of oxidative stress (see FIG. 6). A reduction of oxidative stress leads to the suppression of lipotoxicity, decreased hepatic and stellate cell activation, reduced TGF-β and connective tissue growth factor (CTGF) production. Reduction in TGF-β would also reduce hydrogen peroxide, NF-kB, and apoptosis. It has been shown that oxidative stress on activated human stellate cells leads to increases in the levels of matrix metalloproteinase-1 (TIMP-1) and decreases in the levels of matrix metalloproteinase-1 (MMP). In a human stellate cell line, investigators have shown that H₂O₂ (major component of reactive oxidative species (ROS)) generated by leptin, through activation of ERK1/2 and p38 signaling, inhibited MMP mRNA and promoter activity. Leptin also stimulated TIMP promoter activity and TIMP mRNA expression via by MAP kinase signaling mechanisms. By using a specific ROS inhibitor (catalase, an inhibitor of H₂O₂), it was shown that the opposing expression of both MMP and TIMP are dependent on ROS-mediated downstream signaling processes. Accordingly, it is postulated herein that high-dose niacin, or a niacin analog thereof, will stabilize or normalize the expression levels of MMP and TIMP.

As shown in the results presented herein, high-dose niacin can be used to treat, and even reverse fibrosis. Thus, high-dose niacin, or a niacin analog thereof, can be used to treat diseases, disorders or conditions associated with fibrosis. Fibrotic disease can affect many organs, including liver, bone marrow, lung, kidney, gastrointestinal tract, skin, eye, musculosketal system, and endomyocardium, leading eventually to organ failure. Specific examples of such diseases, disorders and conditions include, but are not limited to, pulmonary fibrosis, such as cystic fibrosis, idiopathic pulmonary fibrosis; radiation-induced lung injury; post-COVID 19 fibrosis; bridging NASH; liver cirrhosis; liver fibrosis; glial scars; arterial stiffness; arthrofibrosis; Crohn's disease, Dupuytren's contracture; Keloids; mediastinal fibrosis; myelofibrosis; Peyronie's disease; nephrogenic system fibrosis; progressive massive fibrosis; retroperitoneal fibrosis; scleroderma/systemic sclerosis; adhesive capsulitis; myocardial fibrosis, such as interstitial fibrosis and replacement fibrosis; inflammatory bowel disease; renal fibrosis in patients with tubulointerstitial fibrosis; glomerulosclerosis; and chronic kidney disease. As shown in the results herein, high-dose niacin reversed hepatic fibrosis, in part by decreasing or degrading excess ECM proteins in cells and tissue. The accumulation of ECM proteins in cells and tissue is a common causation component for most fibrotic conditions and diseases. For example, pulmonary fibrosis is characterized by excessive deposition of collagen and other extracellular matrices (ECM) components as well as the lungs' inability to reconstruct the damaged alveolar epithelium, and persistence of fibroblasts.

In a particular embodiment, the disclosure provides for the reversal or regressing of fibrosis in a subject who has been diagnosed with liver fibrosis and/or liver cirrhosis by using invasive liver biopsies, and/or noninvasive screening of biochemical markers for liver fibrosis or transient elastography. The complete evaluation of a patient with possible liver fibrosis requires clinical evaluation, laboratory tests, and pathological examination. The liver biopsy is regarded as the historical ‘gold standard’ for diagnosis and assessment of prognosis in CLD. Two scoring methods commonly used to stage liver fibrosis are Knodell, and METAVIR. The Knodell and METAVIR score fibrosis from stage 0-4, with stage 4 as cirrhosis. In a particular embodiment, the disclosure provides for administering high-dose niacin (e.g., one or more pharmaceutical doses of niacin) to a subject who has been diagnosed with grade 1, grade 2, grade 3 or grade 4 liver fibrosis. In a further embodiment, the disclosure provides for administering high-dose niacin (e.g., one or more pharmaceutical doses of niacin) to a subject who has been diagnosed with liver cirrhosis.

Over the past years, several noninvasive tests have become available to assess liver fibrosis, primarily in patients with chronic hepatitis C infection. The currently available noninvasive tests, which are surrogate markers of liver fibrosis (direct markers of fibrosis), such as serum hyaluronate, Type IV collagen, matrix metalloproteinase 1 (MMP), tissue inhibitor of matrix metalloproteinase-1 (TIMP-1), laminin, and TGF (3, have limited accuracy for diagnosis of significant fibrosis (METAVIR>F2 or Ishak >3). Other noninvasive tests (indirect markers of liver fibrosis) include FibroTest-ActiTest, APRI, Forns fibrosis index, and enhanced liver fibrosis (ELF) score. The diagnostic performance of these indices is generally good, with a receiver operating characteristics (ROC) curve ranging from 0.77-0.88.

FT-AT, from Biopredictive, Paris, France, is a noninvasive blood test that combines the quantitative results of six serum biochemical markers (alfa2-macroglobulin, haptoglobin, gamma glutamyl transpeptidase, total bilirubin, apolipoprotein A1, and alanine aminotransferase (ALT)) with patients' age and gender in a patented algorithm in order to generate a measure of fibrosis and necroinflammatory activity in the liver. FT-AT provides an accurate measurement of bridging fibrosis and/or moderate necroinflammatory activity with area under the receiver operating curve (AUROC) predictive value between 0.70 and 0.80, when compared to the liver biopsy.

Transient elastography or Fibroscan (Echosens, Paris, France) has become available, which measures liver stiffness or elasticity to assess liver fibrosis. The scan was developed on the principle that livers with increasing degrees of scarring or fibrosis have decreasing elasticity and that a shear wave propagating through stiffer material would progress faster than in one with more elastic material. Transient elastography is painless, rapid, and easily performed at the bedside or in the outpatient clinic. A recent systemic review identified twelve studies, 9 for FibroTest (N=1,679) and 4 for Fibroscan (N=546) and the area under the curve (AUCs) for FibroTest and Fibroscan were 0.90 (95% CI not calculable) and 0.95 (95% CI 0.87-0.99), respectively. The combined use of transient elastography and biochemical markers can help the clinician decide whether a liver biopsy is necessary in some patients, and accordingly decide who to treat with high-dose niacin, or a niacin analog thereof.

While it may be possible for the high-dose niacin, or a niacin analog thereof, to be administered as the raw chemical, it is also possible to present them as a pharmaceutical composition. Accordingly, provided herein are pharmaceutical compositions which comprise high-dose niacin (e.g., one or more pharmaceutical doses of niacin), or a niacin analog thereof, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc., New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of high-dose niacin, or a niacin analog thereof, disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

High-dose niacin (e.g., one or more pharmaceutical doses of niacin), or a niacin analog thereof, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of high-dose niacin, or a niacin analog thereof, to allow for the preparation of highly concentrated solutions.

In addition to the formulations described previously, high-dose niacin, or a niacin analog thereof, may also be formulated as a depot preparation. Such long-acting formulations may be administered by implantation (for example subcutaneously or intramuscularly) or by intramuscular injection. Thus, for example, high-dose niacin, or a niacin analog thereof, may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The pharmaceutical compositions disclosed herein may be formulated as immediate or modified release dosage forms, including delayed, sustained, pulsed, controlled, targeted, timed, and programmed-release forms.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed, extended, prolonged, sustained, timed, pulsatile, controlled, accelerated, rapid, targeted, programmed release forms, and gastric retention dosage forms. The pharmaceutical compositions in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

In one embodiment, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride; and nanoparticles, such as chitosan, and poly-glutamic acid.

In a matrix-controlled release system, the desired release kinetics can be controlled, for example, via the polymer type employed, the polymer viscosity, and the particle sizes of the polymer and/or the active ingredient, the ratio of the active ingredient versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

Preferred unit dosage formulations are those containing an effective dose, as herein below recited, or an appropriate fraction thereof, of the active ingredient.

High-dose niacin, or a niacin analog thereof, may be administered at a dose of 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg or a range that includes or is between any two of the foregoing doses. The total daily dose range for adult humans is generally from 500 mg to 6000 mg. Tablets or other forms of presentation provided in discrete units may conveniently contain an amount of high-dose niacin, or a niacin analog thereof, which is effective at such dosage or as a multiple of the same, for instance, units containing 500 mg to 1000 mg, usually around 500 mg.

The amount of high-dose niacin, or a metabolite or derivative thereof that may be combined with the carrier materials to produce a single dosage form will vary depending upon the subject to be treated and the particular mode of administration.

High-dose niacin, or a niacin analog thereof, can be administered in various modes, e.g., orally, intravenously, transdermally or by injection. The precise amount of high-dose niacin, or a niacin analog thereof, administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of high-dose niacin, or a niacin analog thereof, may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of high-dose niacin, or a niacin analog thereof, may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods to treat or reverse the effects of fibrosis and/or liver cirrhosis in a subject in need thereof, comprising: administering to the subject one or more doses of a pharmaceutical composition comprising 250 mg to 2000 mg of niacin. In a further embodiment, the subject is administered niacin, or a niacin analog thereof, at a total daily dose of 250 mg, 300 mg, 350 mg, 400 mg, 450 mg, 500 mg, 600 mg, 700 mg, 750 mg, 800 mg, 900 mg, 1000 mg, 1100 mg, 1200 mg, 1250 mg, 1300 mg, 1400 mg, 1500 mg, 1600 mg, 1700 mg, 1750 mg, 1800 mg, 1900 mg, 2000 mg, 2100 mg, 2200 mg, 2250 mg, 2300 mg, 2400 mg, 2500 mg, 2600 mg, 2700 mg, 2750 mg, 2800 mg, 2900 mg, 3000 mg, 3100 mg, 3200 mg, 3300 mg, 3400 mg, 3500 mg, 3600 mg, 3700 mg, 3800 mg, 3900 mg, 4000 mg, 4100 mg, 4200 mg, 4300 mg, 4400 mg, 4500 mg, 4600 mg, 4700 mg, 4800 mg, 4900 mg, 5000 mg, 5100 mg, 5200 mg, 5300 mg, 5400 mg, 5500 mg, 5600 mg, 5700 mg, 5800 mg, 5900 mg, 6000 mg, or a range that includes or is between any two of the foregoing values (e.g., 250 mg to 6000 mg). In yet a further embodiment, the subject is administered high-dose niacin, or a niacin analog thereof, for a period of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range that includes or is between any two of the foregoing periods of time. In yet a further embodiment, the subject is administered high-dose niacin, or a niacin analog thereof, for a period of at least 1 week, at least 2 weeks, at least 3 weeks, at least 4 weeks, at least 5 weeks, at least 10 weeks, at least 15 weeks, at least 20 weeks, at least weeks 30 weeks, or at least 52 weeks. Fibrosis can affect various tissues or organs, including but not limited to, liver, bone marrow, lung, kidney, gastrointestinal tract, skin, eye, musculosketal system, endomyocardium, and myocardium. In a particular embodiment, high-dose niacin, or a niacin analog thereof, disclosed herein is used to treat or reverse the effects of fibrosis in the liver of a subject. Fibrosis is associated with many disease, disorders, or conditions including, but not limited to, cystic fibrosis, idiopathic pulmonary fibrosis, radiation-induced lung injury, nonalcoholic steatohepatitis (NASH), liver cirrhosis, glial scars, arterial stiffness, arthrofibrosis, Crohn's disease, Dupuytren's contracture, Keloids, mediastinal fibrosis, myelofibrosis, Peyronie's disease, nephrogenic system fibrosis, progressive massive fibrosis, retroperitoneal fibrosis, scleroderma/systemic sclerosis, adhesive capsulitis, interstitial fibrosis, replacement fibrosis, and Inflammatory Bowel disease, Renal fibrosis in patients with tubulointerstitial fibrosis, glomerulosclerosis, and chronic kidney disease. In a certain embodiment, high-dose niacin, or a niacin analog thereof, disclosed herein is used to treat or reverse the effects of fibrosis in a subject who has NASH or liver cirrhosis.

In a certain embodiment, a method to treat or reverse the effects of fibrosis in a subject in need thereof, comprising: administering to the subject one or more doses of a pharmaceutical composition comprising 500 mg to 1500 mg of niacin, or a niacin analog thereof, so as to cause: (1) regression or reversal of fibrosis; (2) reduction in oxidative stress; (3) reduction in the level of tissue inhibitor metalloproteinase-1 (TIMP-1) and increase in the level of matrix metalloproteinase-1 (MMP); and/or reversal and/or suppression collagen deposition in stellate cells.

High-dose niacin, or a niacin analog thereof, disclosed herein may also be combined or used in combination with other agents useful in the treatment of thrombosis. Or, by way of example only, the therapeutic effectiveness of high-dose niacin, or a niacin analog thereof, described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with high-dose niacin, or a niacin analog thereof, as disclosed herein. When high-dose niacin, or a niacin analog thereof, is used contemporaneously with one or more other drugs, a pharmaceutical composition containing such other drugs in addition to high-dose niacin, or a niacin analog thereof, disclosed herein may be utilized, but is not required.

High-dose niacin, or a niacin analog thereof, disclosed herein is sequentially or concurrently administered in combination with other classes of therapeutics, including, but not limited to, anti-fibrotic therapeutics; antivirals; gallstone solubilizing agents; anti-retroviral agents; CYP3A inhibitors; CYP3A inducers; protease inhibitors; adrenergic agonists; anti-cholinergics; mast cell stabilizers; xanthines; leukotriene antagonists; glucocorticoids treatments; local or general anesthetics; non-steroidal anti-inflammatory agents (NSAIDs), such as naproxen; antibacterial agents, such as amoxicillin; cholesteryl ester transfer protein (CETP) inhibitors, such as anacetrapib; anti-fungal agents, such as isoconazole; sepsis treatments, such as drotrecogin-a; steroidals, such as hydrocortisone; local or general anesthetics, such as ketamine; norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; gallstone solubilizing agents, such as ursodiol; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin; galectin inhibitors TGF-beta inhibitors, anti-TGF-beta antibodies, anti-oxidants, oxidative stress inhibitors, TIMP activators, TIMP inhibitors, MMP activators, MMP inhibitors, kynurenic acid, FS2, cenicriviroc, aramchol, aramchol meglumine, and belapectin.

In another embodiment, high-dose niacin, or a niacin analog thereof, disclosed herein is sequentially or concurrently administered in combination with one or more therapeutics that target lipid metabolism and insulin resistance; one or more therapeutics that target lipotoxicity, oxidative stress, and inflammation; one or more therapeutics that target fibrosis and cirrhosis; or a combination thereof. In a certain embodiment, high-dose niacin, or a niacin analog thereof, disclosed herein can be administered in combination of one or more therapeutics that target lipid metabolism and insulin resistance. Examples of such therapeutics, include but are not limited to, acetyl-CoA carboxylase (ACC) Inhibitors, fatty acid synthase inhibitors, icosbutate, eicosapentaenoic acid analogs/derivatives, omega/n-3 fatty acids, thiazolidinediones (TZDs), 3-hydroxy-3-methylglutaryl-coenzyme A reductase (HMG-CoA reductase) inhibitors, dibrates, peroxisome proliferated-activated receptors (PPAR)-alpha/beta/gamma/delta agonists/modulators, fibroblast growth factor (FGF)-19/21 analogs/modulators, glucagon-like peptide receptors (GLP-1) analogs/mimetics/modulators, ketohexokinase inhibitors, mitochondrial pyruvate carrier inhibitor/modulators, sodium-glucose cotransporters (SGLTs) inhibitors/modulators, adenosine monophosphate-activated protein kinase (AMPK) activators/modulators, stearoyl-CoA dehydrogenase (SCD) inhibitors, diacylglycerol-acyl transferase-2 (DGAT-2) inhibitors, thyroid hormone receptor-beta (THR-beta) agonists, glucocorticoid modulators, dipeptidyl peptidase-4 inhibitors (DPP-4), sodium glucose co-transporter 2 (SGLT2) inhibitors and other anti-diabetes 2 agents. In a further embodiment, high-dose niacin, or a niacin analog thereof, disclosed herein can be administered in combination of one or more therapeutics that target lipotoxicity, oxidative stress, and inflammation. Examples of such therapeutics, include but are not limited to, PPAR-alpha/beta/gamma/delta agonists/modulators, farnesoid X receptor (FXR) agonists/modulators, vitamin E, anti-oxidants, FGF-19/21 modulators, chemokine 2/5 receptor (CCR2/5) antagonists/Inhibitors, nicotinamide adenine dinucleotide phosphate oxidase (NADPH oxidase) inhibitors, and angiotensin receptor antagonists. In yet another embodiment, high-dose niacin, or a niacin analog thereof, disclosed herein can be administered in combination of one or more therapeutics that target fibrosis and cirrhosis. Examples of such therapeutics, include but are not limited to, caspases inhibitors/modulators, apoptosis inhibitors/modulators, leukotriene/phosphodiesterase/lipoxygenase antagonists/inhibitors/modulators, galectin-3 antagonists/modulators, apoptosis signal-regulating kinases (ASK) inhibitors, lysophosphatidic acid receptor 1 (LPA1) antagonists, and heat shock proteins (HSP47 and other members) inhibitors.

In a certain embodiment, high-dose niacin, or a niacin analog thereof, disclosed herein is sequentially or concurrently administered in combination with one or more therapeutics selected from cenicriviroc, resmetirom, ocaliva, elafibranor, aramchol, IMM124E, semaglutide, lanifibranor, seladelpar, belapectin, PXL_065, MADC_0602, aldafermin, VK2809, EDP_305, PF_05221304, tipelukast, tropifexor, DF102, LMB763, nitazoxanide, tesamorelin, TERN_101, lazarotide, BMS986036, Saroglitazar, AKR001, CRV431, GRI_0621, EYP001, BMS_986171, isosabutate, PF_06835919, PF_06865571, nalmefene, LIK066, BIO89_100, Namodenoson, MT_3995, pemafibrate, PXL770, gemcabene, foralumab, SGM_1019, KBP_042, hepastem, CER_209, DUR928, sotagliflozin, elobixibat, SAR425899, NGM313, namacizumab, TERN_201, LPCN_1144, ND_L02_S0201, RTU_1096, IONIS_DGAT2R, bezafibrate, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, and GKT_137831. Thus, in another aspect, certain embodiments provide methods for treating fibrosis-mediated disorders (e.g., liver cirrhosis) in a subject in need of such treatment comprising administering to said subject an amount of high-dose niacin, or a niacin analog thereof, disclosed herein effective to reduce or prevent said disorder in the subject, in combination with at least one additional agent for the treatment of said disorder. In a related aspect, certain embodiments provide therapeutic compositions comprising high-dose niacin, or a niacin analog thereof, disclosed herein in combination with one or more additional agents for the treatment of fibrosis-mediated disorders (e.g., liver cirrhosis).

For use in the therapeutic or biological applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise high-dose niacin disclosed herein, optionally in a composition or in combination with another agent (e.g., antifibrotic therapeutic or a prostaglandin D2 binding drug) as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The disclosure further provides that the methods and compositions described herein can be further defined by the following aspects (aspects 1 to 52):

1. A method to reverse or regress fibrosis and/or liver cirrhosis in a subject in need thereof, comprising:

administering to a subject having fibrosis and/or liver cirrhosis one or more pharmaceutical doses of a pharmaceutical composition comprising niacin, or of a niacin analog thereof, wherein the pharmaceutical composition comprises 250 mg to 2000 mg of niacin, or niacin equivalent dosing of a niacin analog thereof,

wherein the subject is administered a total daily dose of 250 mg to 6000 mg of niacin, or niacin equivalent dosing of a niacin analog thereof, and

wherein administration of the one or more pharmaceutical doses of niacin or a niacin analog thereof reverses or regresses fibrosis and/or liver cirrhosis in the subject.

2. The method of aspect 1, wherein the fibrosis is associated with elevated or overaccumulation of collagen in cells or tissue.

3. The method of aspect 1 or aspect 2, wherein administration of one or more pharmaceutical doses of niacin or of a niacin analog to the subject reduces collagen levels in fibrotic tissue.

4. The method of any one of the preceding aspects, wherein administration of one or more pharmaceutical doses of niacin or of a niacin analog stabilizes or normalizes the expression levels of matrix metalloproteinases (MMPs) and/or tissue inhibitors of metalloproteinases (TIMPs).

5. The method of any one of the preceding aspects, wherein the fibrosis affects one or more tissues or organs.

6. The method of aspect 5, wherein the one or more tissues or organs are selected from liver, bone marrow, lung, kidney, gastrointestinal tract, skin, eye, endomyocardium, musculoskeletal system, and myocardium.

7. The method of aspect 6, wherein the one or more tissues or organs is the liver.

8. The method of any one of the preceding aspects, wherein the subject has a disease, disorder, or condition selected from the group consisting of a cystic fibrosis, idiopathic pulmonary fibrosis, post COVID-19 fibrosis, radiation-induced lung injury, liver fibrosis, liver cirrhosis, glial scars, arterial stiffness, arthrofibrosis, Crohn's disease, Dupuytren's contracture, keloids, mediastinal fibrosis, myelofibrosis, Peyronie's disease, nephrogenic system fibrosis, progressive massive fibrosis, retroperitoneal fibrosis, scleroderma/systemic sclerosis, adhesive capsulitis, interstitial fibrosis, replacement fibrosis, inflammatory bowel disease, renal fibrosis in patients with tubulointerstitial fibrosis, glomerulosclerosis, lung fibrosis, and chronic kidney disease.

9. The method of any one of the preceding aspects, wherein the subject has grade 1, grade 2, grade 3, or grade 4 liver fibrosis.

10. The method of aspect 9, wherein the subject has grade 1, grade 2, or grade 3 liver fibrosis.

11. The method of aspect 9, wherein the subject has grade 4 liver fibroses (i.e., cirrhosis).

12. The method of any one of the preceding aspects, wherein the subject has liver fibrosis and nonalcoholic steatohepatitis, or liver fibrosis and alcoholic steatohepatitis.

13. The method of any one of aspects 1 to 11, wherein the subject has liver fibrosis resulting from a biliary obstruction, iron overload, autoimmune hepatitis, Wilson's disease, a viral hepatitis B infection, or a viral hepatitis C infection.

14. The method of any one of the preceding aspects, wherein the niacin analog is selected from nicotinamide, 6-hydroxy nicotinamide, N-methyl-nicotinamide, acifran, acipimox, niceritrol, ARI-3037MO, and nicotinamide riboside chloride.

15. The method of any one of the preceding aspects, wherein the pharmaceutical composition is formulated for oral, transdermal or parenteral delivery.

16. The method of aspect 15, wherein the pharmaceutical composition is formulated as an extended-release or time-release formulation for oral delivery.

17. The method of aspect 16, wherein the pharmaceutical composition is formulated as a film-coated extended-release tablet.

18. The method of aspect 17, wherein the film-coated extended-release tablet comprises hypromellose, povidone, stearic acid, polyethylene glycol, and/or coloring reagents.

19. The method of aspect 15, wherein the pharmaceutical composition is formulated as a tablet and comprises croscarmellose sodium, hydrogenated vegetable oil, magnesium stearate and/or microcrystalline cellulose.

20. The method of any one of the preceding aspects, wherein the one or more pharmaceutical doses are administered sequentially or concurrently with one or more therapeutics selected from anti-fibrotic therapeutics, prostaglandin D2 binding drugs, antivirals, gallstone solubilizing agents, anti-thrombotic treatments, nonalcoholic fatty liver disease (NAFLD) treatments, nonalcoholic steatohepatitis (NASH) treatments, sepsis treatments, anti-mycobacterial agents, chelation therapy agents, anti-bacterial agents, anti-fungal agents, steroidal drugs, anticoagulants, non-steroidal anti-inflammatory agents, antiplatelet agents, norepinephrine reuptake inhibitors (NRIs), dopamine reuptake inhibitors (DRIs), Serotonin and norepinephrine reuptake inhibitors (SNRIs), sedatives, Norepinephrine and Dopamine Reuptake Inhibitors (NDRIs), serotonin-norepinephrine-dopamine reuptake inhibitors (SNDRIs), monoamine oxidase inhibitors, hypothalamic phospholipids, Endothelin converting enzymes (ECE) inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIa Inhibitors and Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, microsomal triglyceride transfer protein (MTP) Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, anti-inflammatories, anti-proliferatives, chemotherapeutic agents, immunosuppressants, anticancer agents and cytotoxic agents, anti-metabolites, antibiotics, farnesyl-protein transferase inhibitors, hormonal agents, microtubule-disruptor agents, microtubule-stabilizing agents, plant-derived products, epipodophyllotoxins, taxanes, topoisomerase inhibitors, prenyl-protein transferase inhibitors, cyclosporins, cytotoxic drugs, tumor necrosis factor (TNF)-alpha inhibitors, anti-TNF antibodies and soluble TNF receptors, cyclooxygenase-2 (COX-2) inhibitors, galectin inhibitors, transforming growth factor (TGF)-beta inhibitors, anti-TGF-beta antibodies, anti-oxidants, oxidative stress inhibitors, TIMP inhibitors, matrix metalloproteinase-1 (MMP) activators, kynurenic acid, FS2, cenicriviroc, aramchol, aramchol meglumine, and belapectin.

21. The method of aspect 20, wherein the one or more pharmaceutical doses are administered sequentially or concurrently with one or more anti-fibrotic therapeutics.

22. The method of aspect 21, wherein the one or more anti-fibrotic therapeutics are selected from nintedanib, pirfenidone, rilonacept, tocilizumab, rituximab, abatacept, lanifibranor, NCT02503644, NCT03597933, FCX-103, and SAR100842.

23. The method of aspect 20, wherein the one or more pharmaceutical doses is administered sequentially or concurrently with a prostaglandin D2 binding drug.

24. The method of aspect 23, wherein the prostaglandin D2 binding drug is laropiprant.

25. The method of aspect 20, wherein the one or more pharmaceutical doses are administered sequentially or concurrently with one or more therapeutics selected from NASH treatments, NAFLD treatments, antiviral drugs, and gallstone solubilizing agents.

26. The method of aspect 25, wherein the NASH treatments and NAFLD treatments are selected from orlistat, elafibranor, pioglitazone, saroglitazar, solithromycin, exenatide, liraglutide, sitagliptin, vildapliptin, aramchol, obeticholic acid, cenicriviroc, pentoxifylline, emricasan, simtuzumab, galectin-3, atorvastatin, pravastatin, cerivastatin, lovastatin, mevastatin, pitavastatin, rosuvastatin, simvastatin, fluvastatin, NGM-282, GS-4997, IMM-124e, cysteamine, cystamine, and vitamin E.

27. The method of any one of the preceding aspects, wherein the one or more pharmaceutical doses are administered sequentially or concurrently with one or more therapeutics that target lipid metabolism and insulin resistance; one or more therapeutics that target lipotoxicity, oxidative stress, and inflammation; one or more therapeutics that target fibrosis and cirrhosis; or a combination thereof.

28. The method of aspect 27, wherein the one or more pharmaceutical doses are administered sequentially or concurrently with one or more therapeutics selected from acetyl-CoA carboxylase (ACC) Inhibitors, fatty acid synthase inhibitors, icosbutate, eicosapentaenoic acid analogs/derivatives, omega/n-3 fatty acids, thiazolidinediones (TZDs), 3-hydroxy-3-methylglutaryl-coenzyme A reductase (HMG-CoA reductase) inhibitors, dibrates, peroxisome proliferated-activated receptors (PPAR)-alpha/beta/gamma/delta agonists/modulators, fibroblast growth factor (FGF)-19/21 analogs/modulators, glucagon-like peptide receptors (GLP-1) analogs/mimetics/modulators, ketohexokinase inhibitors, mitochondrial pyruvate carrier inhibitor/modulators, sodium-glucose cotransporters (SGLTs) inhibitors/modulators, adenosine monophosphate-activated protein kinase (AMPK) activators/modulators, stearoyl-CoA dehydrogenase (SCD) inhibitors, diacylglycerol-acyl transferase-2 (DGAT-2) inhibitors, thyroid hormone receptor-beta (THR-beta) agonists, glucocorticoid modulators, dipeptidyl peptidase-4 inhibitors (DPP-4), sodium glucose co-transporter 2 (SGLT2) inhibitors, anti-diabetes 2 agents, PPAR-alpha/beta/gamma/delta agonists/modulators, farnesoid X receptor (FXR) agonists/modulators, vitamin E, anti-oxidants, FGF-19/21 modulators, chemokine 2/5 receptor (CCR2/5) antagonists/Inhibitors, nicotinamide adenine dinucleotide phosphate oxidase (NADPH oxidase) inhibitors, and angiotensin receptor antagonists, caspases inhibitors/modulators, apoptosis inhibitors/modulators, leukotriene/phosphodiesterase/lipoxygenase antagonists/inhibitors/modulators, galectin-3 antagonists/modulators, apoptosis signal-regulating kinases (ASK) inhibitors, lysophosphatidic acid receptor 1 (LPA1) antagonists, and heat shock proteins (HSP47 and other members) inhibitors.

29. The method of any one of the preceding aspects, wherein the one or more pharmaceutical doses are administered sequentially or concurrently with one or more therapeutics selected from laropiprant, cenicriviroc, resmetirom, ocaliva, elafibranor, aramchol, IMM124E, semaglutide, lanifibranor, seladelpar, belapectin, PXL_065, MADC_0602, aldafermin, VK2809, EDP_305, PF_05221304, tipelukast, tropifexor, DF102, LMB763, nitazoxanide, tesamorelin, TERN_101, lazarotide, BMS986036, Saroglitazar, AKR001, CRV431, GRI_0621, EYP001, BMS_986171, isosabutate, PF_06835919, PF_06865571, nalmefene, LIK066, BIO89_100, Namodenoson, MT_3995, pemafibrate, PXL770, gemcabene, foralumab, SGM_1019, KBP_042, hepastem, CER_209, DUR928, sotagliflozin, elobixibat, SAR425899, NGM313, namacizumab, TERN_201, LPCN_1144, ND_L02_S0201, RTU_1096, IONIS_DGAT2R, bezafibrate, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, and GKT_137831.

30. The method of any one of the preceding aspects, wherein the subject is administered a total daily dose of 1000 mg to 3000 mg of niacin, or niacin equivalent dosing of a niacin analog thereof.

31. A method to suppress or inhibit accumulation of extracellular matrix components in a subject's cells due to inflammation, comprising:

administering to the subject one or more pharmaceutical doses of a pharmaceutical composition comprising niacin, or of a niacin analog thereof, wherein the composition comprises 250 mg to 2000 mg of niacin, or niacin equivalent dosing of a niacin analog thereof,

wherein the subject is administered a total daily dose of 250 mg to 6000 mg of niacin, or niacin equivalent dosing of a niacin analog thereof, and

wherein administration of the one or more pharmaceutical doses of niacin or a niacin analog thereof suppresses or inhibits accumulation of extracellular matrix components in a subject's cells.

32. The method of aspect 31, wherein the subject has chronic inflammation.

33. The method of aspect 32, wherein the chronic inflammation is caused by exposure to environmental toxins or pollutants, alcohol abuse, radiation treatment, medications, medical conditions, immune diseases or disorders, cholestatic disorders, inherited metabolic disorders, and infectious agents.

34. The method of any one of aspects 31 to 33, wherein the subject's cells are hepatic stellate cells.

35. The method of any one of aspects 31 to 34, wherein the extracellular matrix components comprise collagen.

36. The method of any one of aspects 31 to 35, wherein the niacin analog is selected from nicotinamide, 6-hydroxy nicotinamide, N-methyl-nicotinamide, acifran, acipimox, niceritrol, ARI-3037M0, and nicotinamide riboside chloride.

37. The method of any one of aspects 31 to 36, wherein the pharmaceutical composition is formulated for oral, transdermal or parenteral delivery.

38. The method of aspect 37, wherein the pharmaceutical composition is formulated as an extended-release or time-release formulation for oral delivery.

39. The method of aspect 38, wherein the pharmaceutical composition is formulated as a film-coated extended-release tablet.

40. The method of aspect 39, wherein the film-coated extended-release tablet comprises hypromellose, povidone, stearic acid, polyethylene glycol, and/or coloring reagents.

41. The method of aspect 37, wherein the pharmaceutical composition is formulated as a tablet and comprises croscarmellose sodium, hydrogenated vegetable oil, magnesium stearate and/or microcrystalline cellulose.

42. The method of any one of aspects 31 to 41, wherein the one or more pharmaceutical doses are administered sequentially or concurrently with one or more therapeutics selected from anti-fibrotic therapeutics, prostaglandin D2 binding drugs, antivirals, gallstone solubilizing agents, anti-thrombotic treatments, nonalcoholic fatty liver disease (NAFLD) treatments, nonalcoholic steatohepatitis (NASH) treatments, sepsis treatments, anti-mycobacterial agents, chelation therapy agents, anti-bacterial agents, anti-fungal agents, steroidal drugs, anticoagulants, non-steroidal anti-inflammatory agents, antiplatelet agents, norepinephrine reuptake inhibitors (NRIs), dopamine reuptake inhibitors (DRIs), Serotonin and norepinephrine reuptake inhibitors (SNRIs), sedatives, Norepinephrine and Dopamine Reuptake Inhibitors (NDRIs), serotonin-norepinephrine-dopamine reuptake inhibitors (SNDRIs), monoamine oxidase inhibitors, hypothalamic phospholipids, Endothelin converting enzymes (ECE) inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIa Inhibitors and Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, microsomal triglyceride transfer protein (MTP) Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, anti-inflammatories, anti-proliferatives, chemotherapeutic agents, immunosuppressants, anticancer agents and cytotoxic agents, anti-metabolites, antibiotics, farnesyl-protein transferase inhibitors, hormonal agents, microtubule-disruptor agents, microtubule-stabilizing agents, plant-derived products, epipodophyllotoxins, taxanes, topoisomerase inhibitors, prenyl-protein transferase inhibitors, cyclosporins, cytotoxic drugs, tumor necrosis factor (TNF)-alpha inhibitors, anti-TNF antibodies and soluble TNF receptors, cyclooxygenase-2 (COX-2) inhibitors, galectin inhibitors, transforming growth factor (TGF)-beta inhibitors, anti-TGF-beta antibodies, anti-oxidants, oxidative stress inhibitors, TIMP inhibitors, matrix metalloproteinase-1 (MMP) activators, kynurenic acid, FS2, cenicriviroc, aramchol, aramchol meglumine, and belapectin.

43. The method of aspect 42, wherein the one or more pharmaceutical doses are administered sequentially or concurrently with one or more anti-fibrotic therapeutics.

44. The method of aspect 43, wherein the one or more anti-fibrotic therapeutics are selected from nintedanib, pirfenidone, rilonacept, tocilizumab, rituximab, abatacept, lanifibranor, NCT02503644, NCT03597933, FCX-103, and SAR100842.

45. The method of aspect 42, wherein the one or more pharmaceutical doses is administered sequentially or concurrently with a prostaglandin D2 binding drug.

46. The method of aspect 45, wherein the prostaglandin D2 binding drug is laropiprant.

47. The method of aspect 42, wherein the one or more pharmaceutical doses are administered sequentially or concurrently with one or more therapeutics selected from NASH treatments, NAFLD treatments, antiviral drugs, and gallstone solubilizing agents.

48. The method of aspect 47, wherein the NASH treatments and NAFLD treatments are selected from orlistat, elafibranor, pioglitazone, saroglitazar, solithromycin, exenatide, liraglutide, sitagliptin, vildapliptin, aramchol, obeticholic acid, cenicriviroc, pentoxifylline, emricasan, simtuzumab, galectin-3, atorvastatin, pravastatin, cerivastatin, lovastatin, mevastatin, pitavastatin, rosuvastatin, simvastatin, fluvastatin, NGM-282, GS-4997, IMM-124e, cysteamine, cystamine, and vitamin E.

49. The method of any one of aspects 31 to 48, wherein the one or more pharmaceutical doses are administered sequentially or concurrently with one or more therapeutics that target lipid metabolism and insulin resistance; one or more therapeutics that target lipotoxicity, oxidative stress, and inflammation; one or more therapeutics that target fibrosis and cirrhosis; or a combination thereof.

50. The method of aspect 49, wherein the one or more pharmaceutical doses are administered sequentially or concurrently with one or more therapeutics selected from acetyl-CoA carboxylase (ACC) Inhibitors, fatty acid synthase inhibitors, icosbutate, eicosapentaenoic acid analogs/derivatives, omega/n-3 fatty acids, thiazolidinediones (TZDs), 3-hydroxy-3-methylglutaryl-coenzyme A reductase (HMG-CoA reductase) inhibitors, dibrates, peroxisome proliferated-activated receptors (PPAR)-alpha/beta/gamma/delta agonists/modulators, fibroblast growth factor (FGF)-19/21 analogs/modulators, glucagon-like peptide receptors (GLP-1) analogs/mimetics/modulators, ketohexokinase inhibitors, mitochondrial pyruvate carrier inhibitor/modulators, sodium-glucose cotransporters (SGLTs) inhibitors/modulators, adenosine monophosphate-activated protein kinase (AMPK) activators/modulators, stearoyl-CoA dehydrogenase (SCD) inhibitors, diacylglycerol-acyl transferase-2 (DGAT-2) inhibitors, thyroid hormone receptor-beta (THR-beta) agonists, glucocorticoid modulators, dipeptidyl peptidase-4 inhibitors (DPP-4), sodium glucose co-transporter 2 (SGLT2) inhibitors, anti-diabetes 2 agents, PPAR-alpha/beta/gamma/delta agonists/modulators, farnesoid X receptor (FXR) agonists/modulators, vitamin E, anti-oxidants, FGF-19/21 modulators, chemokine 2/5 receptor (CCR2/5) antagonists/Inhibitors, nicotinamide adenine dinucleotide phosphate oxidase (NADPH oxidase) inhibitors, and angiotensin receptor antagonists, caspases inhibitors/modulators, apoptosis inhibitors/modulators, leukotriene/phosphodiesterase/lipoxygenase antagonists/inhibitors/modulators, galectin-3 antagonists/modulators, apoptosis signal-regulating kinases (ASK) inhibitors, lysophosphatidic acid receptor 1 (LPA1) antagonists, and heat shock proteins (HSP47 and other members) inhibitors.

51. The method of any one of aspects 31 to 49, wherein the one or more pharmaceutical doses are administered sequentially or concurrently with one or more therapeutics selected from laropiprant, cenicriviroc, resmetirom, ocaliva, elafibranor, aramchol, IMM124E, semaglutide, lanifibranor, seladelpar, belapectin, PXL_065, MADC_0602, aldafermin, VK2809, EDP_305, PF_05221304, tipelukast, tropifexor, DF102, LMB763, nitazoxanide, tesamorelin, TERN_101, lazarotide, BMS986036, Saroglitazar, AKR001, CRV431, GRI_0621, EYP001, BMS_986171, isosabutate, PF_06835919, PF_06865571, nalmefene, LIK066, BIO89_100, Namodenoson, MT_3995, pemafibrate, PXL770, gemcabene, foralumab, SGM_1019, KBP_042, hepastem, CER_209, DUR928, sotagliflozin, elobixibat, SAR425899, NGM313, namacizumab, TERN_201, LPCN_1144, ND_L02_S0201, RTU_1096, IONIS_DGAT2R, bezafibrate, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, and GKT_137831.

52. The method of any one of the aspects 31 to 51, wherein the subject is administered a total daily dose of 1000 mg to 3000 mg of niacin, or niacin equivalent dosing of a niacin analog thereof.

The following examples are intended to illustrate but not limit the disclosure. While they are typical of those that might be used, other procedures known to those skilled in the art may alternatively be used.

### EXAMPLES

Human Donor Subjects' Demographics, Medical History, and Liver Histology: Cryopreserved primary cultures of human hepatic stellate cells (HSC, passage 0) isolated from the livers of human subjects without fibrosis or NASH features and patients with fibrosis with associated NASH features steatosis, inflammation) were purchased from Samsara Sciences, Inc., San Diego, Calif. (now available from LifeNet Health). Detailed donor subjects' history report including donor demographic characteristics, cause of death, medical history, and liver pathology provided by Samsara Sciences are summarized in Table 1. In brief, donor control patients (non-fibrosis subjects) past medical histories did not present any major metabolic abnormalities commonly associated with hepatic fibrosis or NASH. Additionally, liver histological assessment performed in liver samples in these normal donor subjects by a certified pathologist showed NAFLD activity score of 0, steatosis grade 0, inflammation score 0, and fibrosis score 0.

TABLE 1
Human Donor Subjects Demographics, Medical History, and Liver Histology

| | Gender, | | | | | | | |
|---|---|---|---|---|---|---|---|---|
| Donor | Race, Age | Weight (kg), | | Cause of | Liver Pathology ** | | | |
| Subjects | (years) | BMI | Past Medical History | Death | NAS* | Steatosis | Inflammation | Fibrosis |
| 1 | Female, | 63.6, 27.4 | Hyperlipidemia | ICH/Stroke | 0 of 8 | 0 of 3 | 0 of 4 | 0 of 4 |
| | Caucasian, | | | | | | | |
| | 59 | | | | | | | |
| 2 | Male, | 72.7, 22.9 | None | Trauma | 0 of 8 | 0 of 3 | 0 of 4 | 0 of 4 |
| | African | | | | | | | |
| | American, | | | | | | | |
| | 24 | | | | | | | |
| 3 | Female, | 87.6, 34.2 | Diabetes type II, | CVA/Stroke | 2 of 8 | 1 of 3 | 1 of 4 | 2-3 of 4 |
| | Hispanic, | | Pancreatitis, high | | | | | |
| | 35 | | cholesterol, fatty liver, | | | | | |
| | | | history of Thyroid cancer | | | | | |
| 4 | Male, | 89.5, 35 | Diabetes Type II, | Anoxia/CVD | 3 of 8 | 1 of 3 | 2 of 4 | 2 of 4 |
| | Hispanic, | | Hypertension, | | | | | |
| | 45 | | Schizophrenia | | | | | |
| 5 | Female, | 127, 48.06 | Hypertension, Diabetes | Cardiac arrest/ | 3 of 8 | 2 of 3 | 1 of 4 | 1-2 of 4 |
| | Caucasian, | | Type II, Depression | Anoxia | | | | |
| | 53 | | | | | | | |
| 6 | Female, | 100, 34.5 | Diabetes Type II, Kidney | Anoxia/ | 4 of 8 | 3 of 3 | 1 of 4 | 1-2 of 4 |
| | Asian, | | disease, Hyper-lipidemia, | Diabetic | | | | |
| | 44 | | Asthma, Arthritis | Ketoacidosis | | | | |
| 7 | Male, | 113, 37.0 | CAD/CABG, | Cardiac arrest/ | 5 of 8 | 2 of 3 | 2 of 4 | 1 of 4 |
| | African | | Hypertension, | Anoxia | | | | |
| | America, | | Hypercholesterolemia | | | | | |
| | 66 | | | | | | | |

*NAS = NAFLD Activity Score: ** NASH CRN Scoring System (Hepatology 41: 1313-1321, 2005)

For the study, the stellate cells were selected from patients who had fibrosis and the features of NASH. The alcohol consumption and remote past history of viral hepatitis was not known. Human donor patients with fibrosis had past histories of varied metabolic abnormalities including type II diabetes, dyslipidemia, hypertension, and coronary artery disease, etc. (see Table 1). Only one patient had a living diagnosis of fatty liver disease. Liver pathology assessment by a certified pathologist revealed varied degree of liver histology in patients with fibrosis including NAFLD activity scores 2-5, steatosis grades 1-3, inflammation scores 1-2, and fibrosis scores 1-3 (see Table 1). The donor subjects ranged in age from 35-66 years, represented both genders, and varied racial origins, including Caucasian, Hispanic, African American, and Asian.

Detailed product information on individual human hepatic cells including their morphology and phenotypic characteristics were previously characterized. Briefly, human normal primary hepatic stellate cells from normal nonfibrotic subject donors were characterized by expression of glial fibrillary acidic protein (GFAP) and to a lesser extent Desmin. Stellate cells from donors with NASH were characterized by activated phenotype with the expression of smooth muscle cell actin (SMA) and collagen. The expression of GFAP, Desmin, SMA were assessed in order to characterize normal and activated stellate cells from control (nonfibrotic cells) human donor subjects and donor subjects with fibrosis. The cultures were also checked for any contamination by fibroblasts or endothelial cells by checking the expression of TE-7 and CD31, respectively.

Preparation of Hepatic Stellate Cells. HSC were isolated based on differential centrifugation through a Nycodenz gradient. In addition to the isolation methods listed above, the stellate cells were further purified using cell culture conditions specific for the cell type. Cryopreserved HSCs were grown in DMEM+10% FBS media containing 1% antibiotic/antimycotic according to the recommended media and procedures. HSCs at passage 2 were used for all in vitro studies described below. During the experimental incubations with niacin, TGF-β or H₂O₂, cells were incubated in DMEM+0.5% FBS.

Hepatic stellate cell fibrosis quantification. Cellular total collagen and collagen type I content, as an index of fibrosis, were measured by commercially available Sirius Red Collagen Detection Kit and Human Type I Collagen Detection Kit (ELISA) respectively from Chondrex, Inc., Redmond, Wash. Cellular digestion with pepsin, solubilization, and assay procedures were performed according to the assay protocols provided in the assay kits. Collagen and Collagen Type I content using Sirius Red and ELISA kits were measured based on the standard curve. Additionally, Sirius Red stained cells were also used for photographic images (magnification 10×) for qualitative visual representation of collagen content in various treatment groups.

Reactive Oxygen Species (ROS) quantification. ROS production, as an index of oxidative stress, in stellate cells was measured using DCFDA fluorescence as described previously. In brief, cells were incubated with DCFDA (10 μmol/L) for 30 min. After washing, the fluorescent intensity in the cell lysate was measured at the excitation and emission wavelength of 488 and 520, respectively. Additionally, DCFDA stained cells were also used for photographic images (magnification 10×) for representation of ROS content.

Assessment of cell viability. Human hepatic cell viability was assessed by using commercially available PrestoBlue Cell Viability Reagent kit by Invitrogen, Carlsbad, Calif. PrestoBlue Reagent kit utilizes a compound that is quickly reduced by metabolically active cells, providing a quantitative measure of viability and cytotoxicity. Cell viability assay and quantitation by measuring fluorescence (at excitation 540-570 nm, emission 580-610 nm) were performed according to the protocol provided in the assay kit by Invitrogen.

Statistical Analysis. Data presented are mean±SE of 3 separate experiments for each subject stellate cells. Statistical significance was calculated by using Student's t test, and a value of p<0.05 was considered significant.

Niacin reverses fibrosis in hepatic stellate cells from human subjects. For these studies, human hepatic stellate cells from donor patients with varying degree of fibrosis (F1-3) and NASH (NAFLD activity scores 2-5) and stellate cells from normal non-fibrosis subjects (NAFLD activity score of 0) were incubated with pharmacologically relevant concentrations of niacin (0.25 mM and 0.5 mM) in DMEM+0.5% media for 48 or 96 hours. Cells were stained with Sirius Red and collagen content quantitated according to the procedure noted in Sirius Red Collagen Detection Kit.

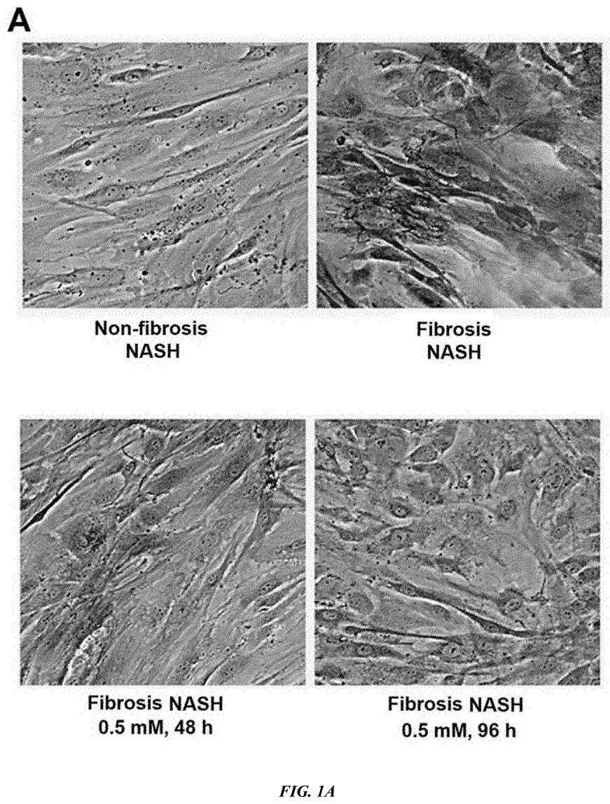

In the representative photographic image of collagen content in stellate cells from non-fibrosis and from fibrotic NASH subjects (see FIG. 1A), collagen content in stellate cells from NASH patient (donor 6) was strikingly higher than in stellate cells from normal non-fibrosis subject (donor 1) which showed very minimal to Sirius Red stainable collagen content. Table 2 displays quantitative changes in collagen content at baseline, 48- and 96-hours incubation with 0.25 mM and 0.50 mM niacin in stellate cells from individual normal non-fibrosis subjects (donors 1, 2) and from patients with fibrosis (donors 3-7). FIG. 1B displays the composite Mean±SE collagen content data from all 5 fibrosis patient (donors 3-7) showing the ability of niacin to regress pre-existing fibrosis in stellate cells from patients with fibrosis. Collagen content in stellate cells from fibrotic patients were strikingly higher (4-fold) than in stellate cells from control non-fibrosis subjects (Table 2, FIG. 1A-B). Treatment of hepatic stellate cells from patients with fibrosis (donor patients 3-7) with niacin (0.25 mM and 0.5 mM) for 48 h or 96 h, respectively, produced a robust and significant regression of pre-existing fibrosis by an average of 47.6 and 60.1 percent (at 48 h incubation) and 53.5 and 65.0% (at 96 h incubation, p<0.001, respectively (Table 2, FIG. 1B). At both 48 and 96 h incubation, 0.5 mM niacin had significantly greater regression of fibrosis than 0.25 mM niacin (60.1 vs 47.6% at 48 h and 65.0 vs 53.5% at 96 h, p<0.03, Table 2, FIG. 1B).

It is striking to note that niacin caused a significant regression of pre-existing fibrosis in stellate cells from all 5 donor subjects. However, niacin did not affect the low measurable collagen content in stellate cells from normal non-fibrosis subjects with NAFLD activity score of 0 and fibrosis score of 0 (Table 2).

TABLE 2
Effect of Niacin on collagen content in hepatic stellate cells from Non-NASH normal Subjects
and NASH patients with fibrosis

| | | Incubation time | | | | | |
|---|---|---|---|---|---|---|---|
| | | 48 h | | | 96 h | | |
| Donor | Niacin | 0 | 0.25 | 0.5 | 0 | 0.25 | 0.5 |
| Subject/Patient | (mM) | Collagen (μg/mL) | | | Collagen (μg/mL) | | |
| Normal Non-Nash | | | | | | | |
| 1 | | 21.5 ± 2.4 | 20.0 ± 0.2 | 20.1 ± 0.1 | 23.4 ± 2.8 | 21.9 ± 1.5 | ND |
| 2 | | 20.4 ± 0.2 | 20.6 ± 0.1 | 24.0 ± 1.1 | 25.2 ± 0.6 | 23.9 ± 0.1 | ND |
| Mean ± SE | | 21.0 ± 1.1 | 20.3 + 0.2 | 22.1 + 1.0 | 24.3 + 1.4 | 22.9 + 0.8 | |
| (Patients 1-2) | | | | | | | |
| NASH with Fibrosis | | | | | | | |
| 3 | | 60.1 ± 1.7 | 32.7 ± 2.7 | 24.6 ± 2.4 | 94.6 ± 5 | 46.4 ± 5.1 | 29.7 ± 5.1 |
| 4 | | 73.3 ± 5.3 | 42.3 ± 2.2 | 28.6 ± 3.2 | 88.4 ± 3.1 | 31.4 ± 2.7 | 21.5 ± 3.2 |
| 5 | | 70.4 ± 5.0 | 24.9 ± 4.5 | 24.3 ± 2.0 | 78.4 ± 7.0 | 22.7 ± 3.6 | 18.8 ± 4.0 |
| 6 | | 96.5 ± 5.6 | 49.2 ± 2.0 | 34.7 ± 2.0 | 98.9 ± 7.6 | 60.7 ± 2.4 | 40.4 ± 4.6 |
| 7 | | 123.1 ± 7.6 | 77.9 ± 6.4 | 60.2 ± 6.8 | 130.3 ± 7.0 | 74.3 ± 7.2 | 70.8 ± 8.8 |
| Mean ± SE | | 84.7 ± 11.3 | 45.4 ± 9.1* | 34.5 ± 6.7* | 98.1 ± 8.7 | 47.1 ± 9.3* | 36.2 ± 9.4* |
| (Patients 3-7) | | | | | | | |
| Mean % Regression | | | 47.6 ± 4.6 | 60.1 ± 2.5 ^{a} | | 53.5 ± 6.2 | 65.0 ± 5.7^{a} |
| (Patients 3-7) | | | | | | | |

*P < 0.001 vs 0 mM Niacin for respective incubation period
^{a} P < 0.03 vs 0.25 mM Niacin for respective incubation period;
ND, Not determined

Niacin prevents TGF-β or H₂O₂-induced fibrosis. Determining whether niacin prevents and reverses stellate cell fibrosis induced by major physiological stimulators of liver fibrosis such as TGF-β or oxidative stress mediator hydrogen peroxide (H₂O₂) was next examined using human hepatic stellate cells from normal non-fibrosis human donor subjects. For these studies, human hepatic stellate cells from non-fibrotic subjects were incubated with TGF-β (20 ng/mL) or H₂O₂ (25 μM) in the absence or presence of niacin (0.5 mM) for 24 h. Hepatic stellate cell fibrosis was assessed by measuring collagen content by Sirius Red staining kit. As shown in the representative photographic images of stellate cells after staining with Sirius Red (FIG. 2, Top panel), incubation of cells with either TGF-β or H₂O₂, known inducers of stellate cell fibrosis, markedly increased collagen content as compared to vehicle treatment (VEH). Co-incubation of these cells with either TGF-β or H₂O₂ and niacin for 24 h noticeably prevented collagen production as shown in cellular photographic images after Sirius Red staining (FIG. 2, Top panel). Treatment of stellate cells from normal non-NASH subjects with niacin (0.5 mM) in the absence of TGF-β or H₂O₂ had no effect on collagen content as shown in photographic image labeled as NIA (FIG. 2, Top panel).

Quantitative data in FIG. 2, bottom bar diagram showed that both H₂O₂ and TGF-β robustly and significantly increased collagen content by 266% and 233% respectively in human stellate cells from non-fibrosis patients. Treatment of these cells with niacin in the presence of either H₂O₂ or TGF-β markedly and significantly prevented collagen production by 59% and 56% respectively when compared to cells treated with H₂O₂ or TGF-β alone (see FIG. 2, bottom bar diagram). Niacin (0.5 mM) in the absence of H₂O₂ or TGF-β had no effect on cellular collagen content (see FIG. 2 bottom bar diagram, last column labeled as NIA 0.5 mM).

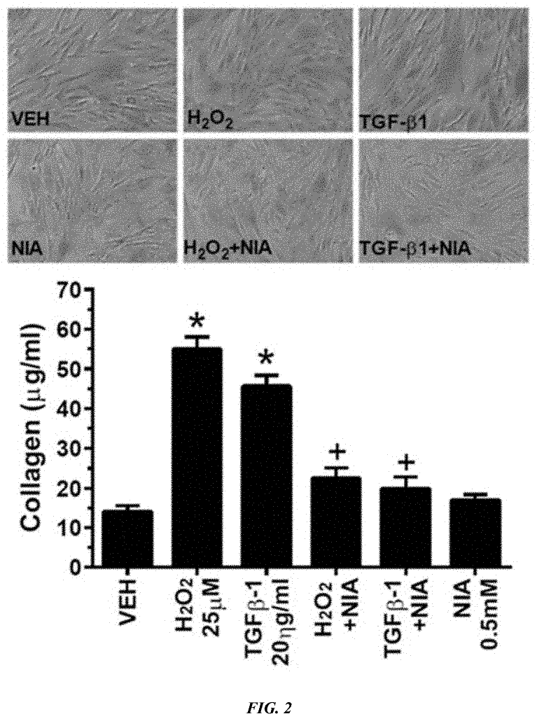

Niacin reverses TGF-β or H₂O₂-induced hepatic stellate cell fibrosis. In these studies, human hepatic stellate cells from non-fibrosis subjects were first stimulated with TGF-β (20 ng/mL) or H₂O₂ (25 μM) for 24 h to induce fibrosis. These cells were then continued to incubate additional 24 h in the absence or presence of niacin (0.5 mM). Cellular content of Collagen type I was measured by ELISA as noted above. As shown in FIG. 3, both H₂O₂ and TGF-β robustly increased by 4-5-fold cellular collagen type I content as compared to vehicle (control). Treatment of these cells with pre-existing fibrosis (induced by H₂O₂ or TGF-β) with niacin (0.5 mM) almost completely reversed cellular fibrosis, and collagen type I contents were like the controls (see FIG. 3). Like FIG. 2 data, treatment of cells with niacin (labeled as Niacin 0.5 mM) without previous stimulation with H₂O₂ or TGF-β had no effect on collagen type I content (see FIG. 3, last column).

Niacin prevents human hepatic stellate cell oxidative stress induced by palmitic acid or H₂O₂. Since oxidative stress with increased cellular ROS plays an important role in hepatic stellate cell fibrosis, the effect of niacin on ROS induced by physiological mediators of oxidative stress such as palmitic acid and H₂O₂ was next investigated. For these studies, human hepatic stellate cells from normal non-fibrosis subjects were first stimulated with either palmitic acid (0.5 mM) or H₂O₂ (25 μM) for 24 h to induce oxidative stress. These cells stimulated with palmitic acid or H₂O₂ were then continued to incubate additional 24 h in the absence or presence of niacin (0.5 mM). Representative cellular photographic images after staining with DCFDA (see FIG. 4 upper panel) showed that both palmitic acid (PA) and H₂O₂ dramatically induced stellate cell ROS levels by 3.6- and 5.2-fold respectively, compared to vehicle (VEH) treatment (quantitatively shown in FIG. 4 lower panel). Treatment of these cells with pre-existing oxidative stress (induced by palmitic acid or H₂O₂) with niacin (0.5 mM) significantly and visibly decreased cellular ROS levels by 52% and 50% respectively (see FIG. 4 lower panel). Treatment of cells with niacin (labeled as NIA) without previous stimulation with palmitic acid or H₂O₂ had no effect on baseline low ROS levels.

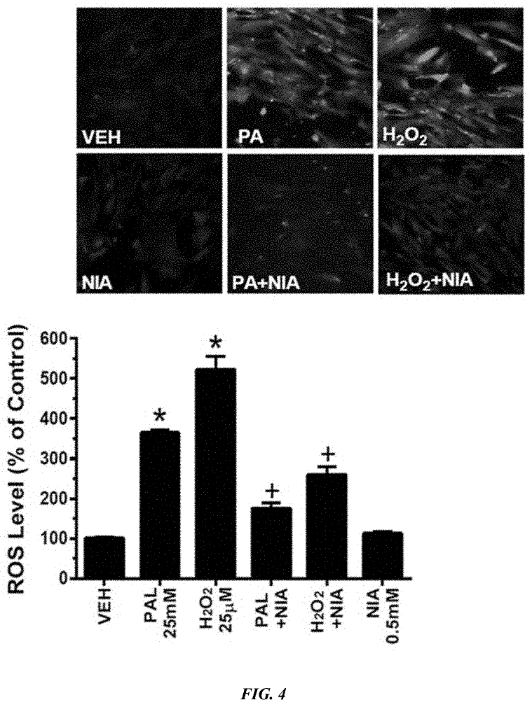

Niacin did not affect human hepatic stellate cell viability: For these studies, human hepatic stellate cells were incubated with niacin (0-0.5 mM) for 48 h or 96 h. Cellular viability was measured by a cell viability assay kit as noted above. As shown in FIG. 5, treatment of cells with niacin (0.25 mM or 0.5 mM) for 48 h and 96 h had no significant effect on cell viability as compared to stellate cells without incubation with niacin.

A number of embodiments have been described herein. Nevertheless, it will be understood that various modifications may be made without departing from the spirit and scope of this disclosure. Accordingly, other embodiments are within the scope of the following claims.

What is claimed is:

1. A method to reverse or regress fibrosis and/or liver cirrhosis in a subject in need thereof, comprising:
administering to a subject having fibrosis and/or liver cirrhosis one or more pharmaceutical doses of a pharmaceutical composition comprising niacin, or of a niacin analog thereof, wherein the pharmaceutical composition comprises 250 mg to 2000 mg of niacin, or niacin equivalent dosing of a niacin analog thereof,
wherein the subject is administered a total daily dose of 250 mg to 6000 mg of niacin, or niacin equivalent dosing of a niacin analog thereof, and
wherein administration of the one or more pharmaceutical doses of niacin or a niacin analog thereof reverses or regresses fibrosis and/or liver cirrhosis in the subject.

2. The method of claim 1, wherein the fibrosis is associated with elevated or overaccumulation of collagen in cells or tissue.

3. The method of claim 2, wherein administration of one or more pharmaceutical doses of niacin or of a niacin analog to the subject reduces collagen levels in fibrotic tissue.

4. The method of claim 1, wherein administration of one or more pharmaceutical doses of niacin or of a niacin analog stabilizes or normalizes the expression levels of matrix metalloproteinases (MMPs) and/or tissue inhibitors of metalloproteinases (TIMPs).

5. The method of claim 1, wherein the fibrosis affects one or more tissues or organs.

6. The method of claim 5, wherein the one or more tissues or organs are selected from liver, bone marrow, lung, kidney, gastrointestinal tract, skin, eye, endomyocardium, musculoskeletal system, and myocardium.

7. The method of claim 6, wherein the one or more tissues or organs is the liver.

8. The method of claim 1, wherein the subject has a disease, disorder, or condition selected from the group consisting of a cystic fibrosis, idiopathic pulmonary fibrosis, post COVID-19 fibrosis, radiation-induced lung injury, liver fibrosis, liver cirrhosis, glial scars, arterial stiffness, arthrofibrosis, Crohn's disease, Dupuytren's contracture, keloids, mediastinal fibrosis, myelofibrosis, Peyronie's disease, nephrogenic system fibrosis, progressive massive fibrosis, retroperitoneal fibrosis, scleroderma/systemic sclerosis, adhesive capsulitis, interstitial fibrosis, replacement fibrosis, inflammatory bowel disease, renal fibrosis in patients with tubulointerstitial fibrosis, glomerulosclerosis, lung fibrosis, and chronic kidney disease.

9. The method of claim 1, wherein the subject has grade 1, grade 2, grade 3, or grade 4 liver fibrosis.

10. The method of claim 1, wherein the subject has liver cirrhosis.

11. The method of claim 1, wherein the subject has liver fibrosis resulting from a biliary obstruction, iron overload, autoimmune hepatitis, Wilson's disease, a viral hepatitis B infection, or a viral hepatitis C infection.

12. The method of claim 1, wherein the niacin analog is selected from nicotinamide, 6-hydroxy nicotinamide, N-methyl-nicotinamide, acifran, acipimox, niceritrol, ARI-3037MO, and nicotinamide riboside chloride.

13. The method of claim 1, wherein the pharmaceutical composition is formulated for oral, transdermal or parenteral delivery.

14. The method of claim 13, wherein the pharmaceutical composition is formulated as an extended-release or time-release formulation for oral delivery.

15. The method of claim 14, wherein the pharmaceutical composition is formulated as a film-coated extended-release tablet.

16. The method of claim 15, wherein the film-coated extended-release tablet comprises hypromellose, povidone, stearic acid, polyethylene glycol, and/or coloring reagents.

17. The method of claim 13, wherein the pharmaceutical composition is formulated as a tablet and comprises croscarmellose sodium, hydrogenated vegetable oil, magnesium stearate and/or microcrystalline cellulose.

18. The method of claim 1, wherein the one or more pharmaceutical doses are administered sequentially or concurrently with one or more therapeutics selected from anti-fibrotic therapeutics, prostaglandin D2 binding drugs, antivirals, gallstone solubilizing agents, anti-thrombotic treatments, nonalcoholic fatty liver disease (NAFLD) treatments, nonalcoholic steatohepatitis (NASH) treatments, sepsis treatments, anti-mycobacterial agents, chelation therapy agents, anti-bacterial agents, anti-fungal agents, steroidal drugs, anticoagulants, non-steroidal anti-inflammatory agents, antiplatelet agents, norepinephrine reuptake inhibitors (NRIs), dopamine reuptake inhibitors (DRIs), Serotonin and norepinephrine reuptake inhibitors (SNRIs), sedatives, Norepinephrine and Dopamine Reuptake Inhibitors (NDRIs), serotonin-norepinephrine-dopamine reuptake inhibitors (SNDRIs), monoamine oxidase inhibitors, hypothalamic phospholipids, Endothelin converting enzymes (ECE) inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIa Inhibitors and Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, microsomal triglyceride transfer protein (MTP) Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, anti-inflammatories, anti-proliferatives, chemotherapeutic agents, immunosuppressants, anticancer agents and cytotoxic agents, anti-metabolites, antibiotics, farnesyl-protein transferase inhibitors, hormonal agents, microtubule-disruptor agents, microtubule-stabilizing agents, plant-derived products, epipodophyllotoxins, taxanes, topoisomerase inhibitors, prenyl-protein transferase inhibitors, cyclosporins, cytotoxic drugs, tumor necrosis factor (TNF)-alpha inhibitors, anti-TNF antibodies and soluble TNF receptors, cyclooxygenase-2 (COX-2) inhibitors, galectin inhibitors, transforming growth factor (TGF)-beta inhibitors, anti-TGF-beta antibodies, anti-oxidants, oxidative stress inhibitors, TIMP inhibitors, matrix metalloproteinase-1 (MMP) activators, kynurenic acid, FS2, cenicriviroc, aramchol, aramchol meglumine, and belapectin.

19. The method of claim 18, wherein the one or more pharmaceutical doses are administered sequentially or concurrently with one or more anti-fibrotic therapeutics.

20. The method of claim 1, wherein the one or more pharmaceutical doses are administered sequentially or concurrently with one or more therapeutics selected from laropiprant, cenicriviroc, resmetirom, ocaliva, elafibranor, aramchol, IMM124E, semaglutide, lanifibranor, seladelpar, belapectin, PXL_065, MADC_0602, aldafermin, VK2809, EDP_305, PF_05221304, tipelukast, tropifexor, DF102, LMB763, nitazoxanide, tesamorelin, TERN_101, lazarotide, BMS986036, Saroglitazar, AKR001, CRV431, GRI_0621, EYP001, BMS_986171, isosabutate, PF_06835919, PF_06865571, nalmefene, LIK066, BIO89_100, Namodenoson, MT_3995, pemafibrate, PXL770, gemcabene, foralumab, SGM_1019, KBP_042, hepastem, CER_209, DUR928, sotagliflozin, elobixibat, SAR425899, NGM313, namacizumab, TERN_201, LPCN_1144, ND_L02_S0201, RTU_1096, IONIS_DGAT2R, bezafibrate, INT_767, NP160, NEULIV, NP135, BFKB8488A, NC_001, VK0214, HM15211, CM_101, AZD2693, NV556, SP_1373, RLBN1127, RYI_018, NVP022, VPR_423, CB4209-CB4211, and GKT_137831.
